SEQUENCE LISTING (200 sequences)

SEQ ID NO: 1 - DNA, 195 bp, Trypanosoma cruzi
ctgtttcgaattatgaatggtggtggtcagagacactctctgtcaatgtctgtttgcgtgttcacacactggacaccaaacaaccctgaattatccgctgcttggaggaatttcgcgagatcatgcccacaggggtgctgcactcggcggatcgttttcgagcggctgctgcaccacacgttgtggtctaaattt

SEQ ID NO: 2 - DNA, 126 bp, Trypanosoma cruzi
gagagtgttgacgccgtggctgccatggagcgccagctacaggagcgtgatgatgccctggctgcgctgaaggacaggctggaggagtacagcagagaaaaatctgcgctggagagccgcaccagt

SEQ ID NO: 3 - DNA, 42 bp, Trypanosoma cruzi
gacgccgtggcggagaatgcccagctgcagaaggagagggat

SEQ ID NO: 4 - DNA, 480 bp, Trypanosoma cruzi
agcgcgagcaccgcctgggtctggatcacgacgaggcgcagccgttggaggccgccctgcttgaggacgaggcgccgcatgcggttgttgagcgacagcgcgagcaccgcctgggtctggatcacgacgaggcgcagccgttggaggccgccctgcttgaggacgaggcgccgcatgcggttgttgagcgacagcgcgagcaccgcctgggtctggatcacgacgaggcgcagccgttggaggccgccctgcttgaggacgaggcgccgcatgcggttgttgagcgacagcgcgagcaccgcctgggtctggagcacgacgaggcgcagccgttggaggccgccctgcttgaggacgaggcgccgcatgcggttgttgagcgacagcgcgagcaccgcctgggtctggatcacgacgaggcgcagccgttggaggccgccctgcttgaggacgaggcgccgcatgcggttgttgagcgac

SEQ ID NO: 5 - DNA, 45 bp, Trypanosoma cruzi
agtggagttggcagcttcatgagcgagggttcgtccaaggaacga

SEQ ID NO: 6 - DNA, 126 bp, Trypanosoma cruzi
cttgcgcagagggaggctgacaatgagaagctcgcagaggaacttgcgcagagggaggctgacaatgagaagctcgcagaggaacttgcgcagagggaggctgacaatgagaagctcgcagaggat

SEQ ID NO: 7 - DNA, 72 bp, Trypanosoma cruzi
ctgcagcgtcaaaatgaggagttgcagtcgcagctgaaggagtcgcgtcgtggagaggagaagctggacgcc

SEQ ID NO: 8 - DNA, 105 bp, Trypanosoma cruzi
gagaacgaggagctgcgtgctgagcacgaacataagacccgtggactgcaggaggtgagcgagcaggcggaggatctgcagcggcagctggaggagcttcgtgcg

SEQ ID NO: 9 - DNA, 126 bp, Trypanosoma cruzi
ccggagacagcctcagtcccagaggaacaccgcggctcgaggaaggtttccggtgcagtgcccgagggcgaggaatatccgactgaggccgagagacctgttgccaccgacgaggacggtcacgcg

SEQ ID NO: 10 - DNA, 30 bp, Trypanosoma cruzi
gccagacggaaggctgcggaggaagaagct

SEQ ID NO: 11 - DNA, 318 bp, Trypanosoma cruzi
tggcaggaacaggagcgtgagcggagggcgaaggaggccgaggaggcgaagcggcagctggagcaggaactgaaggcccagcaggaggcgcgcgaggcggaggagcgccggcgcgtcgagcttgccaagcagcagcgtgaggagtcgaaggcgcgcaaggaggaactgcagcggaagcaggccgaggagaggagaaagaagaaggaggagctgcaggccgagaccgagaggctgctggccgaggcccgcagcgccgaggagggcgagaagaaggcgctcgccgagaaggtgcggacaggaaaggaggaaggcgccagg

SEQ ID NO: 12 - DNA, 27 bp, Trypanosoma cruzi
gaagcagaagcacgccgcctggctgag

SEQ ID NO: 13 - DNA, 117 bp, Trypanosoma cruzi
cagctggacgaggccgtgcagcagcgcgaggaggtgcagcgcgagctggagcgaaccatcgaggaactggccgctctccgggagcagagcggggtggacaccgtgaatctgcgtgcg

SEQ ID NO: 14 - DNA, 117 bp, Trypanosoma cruzi
atggcaacggacgagttgacgacggaggcgaggccgcttgacgtgactgtcggttccgcgctgctcagtgactcggcggcagtaactgcccccgcggacagggatgcgactgcgctc

SEQ ID NO: 15 - DNA, 60 bp, Trypanosoma cruzi
acaacacagccgttgggaggcacgggtgtgaccacggcaacgacacagccattctcggcc

SEQ ID NO: 16 - DNA, 42 bp, Trypanosoma cruzi
ggctggcggatgagcttgagcagaaggcagcggagaatgaga

SEQ ID NO: 17 - DNA, 24 bp, Trypanosoma cruzi
gaggctgagaagcgtgctcaacga

SEQ ID NO: 18 - DNA, 90 bp, Trypanosoma cruzi
gaggatgttcaggaattggtgaccccggctgaggatgttcaggaactggtggctccggctgaggatattcaggaactggtggctccggct

SEQ ID NO: 19 - DNA, 42 bp, Trypanosoma cruzi
aggagagggatgacgccgtggcggagaatgcccagctgcaga

SEQ ID NO: 20 - DNA, 372 bp, Trypanosoma cruzi
acgacctccaagagcgtgctgctgctgccgaggatgccgctcgccgtcgctgtgccgctgcaagggagaaggaggaggctgcgaagcgcctggaggcggagctggaggaacgcacgaacgacctccaagagcgtgctgctgctgccgaggatgccgctcgccgtcgctgtgccgctgcaagggagaaggaggaggctgcgaagcgcctggaggcggagctggaggtgcgcacgaacgacctccaagagcgtgcaaacgatctccaagagcgtgctgctgctgccgaggatgccgctcgccgtcgctgtgccgctgcaagggagaaggaggaggctgcgaagcgcctggaggcggagctggaggagcgcacga

SEQ ID NO: 21 - DNA, 120 bp, Trypanosoma cruzi
gattccgaaaatcctatgtcgggagctgccgcaagtagcatcttggttaaaggggccggcaacagcagctccacgaacacaatgcggaacatgcggaatgcttccgtggcggtccaagag

SEQ ID NO: 22 - DNA, 84 bp, Trypanosoma cruzi
gtgaccgctgagcgcgaggagcttgccgaaaacctgcgggccacggaggacgcgaaggccgaggtggagaggaacctcgagtcc

SEQ ID NO: 23 - DNA, 195 bp, Trypanosoma cruzi
gccgctcgcggtcagctagtcggcgaggagcgcgagggcacgtgtggattgcaccgcgatgccgtggattccgaggagagagcggtgcgccgctgcctggagcgcggcgaggctgcagctgtggacgagctcggagaggagtacgggagcgcgacgcacgagcgagtagtggaggcgctagccgcggaggaggac

SEQ ID NO: 24 - DNA, 42 bp, Trypanosoma cruzi
ggcttcggctctgccacgcacacgtcggcacctgctgctggt

SEQ ID NO: 25 - DNA, 54 bp, Trypanosoma cruzi
tcacccaccttaacggagaccgagaaagggacgggcactccagctcacactgcg

SEQ ID NO: 26 - DNA, 117 bp, Trypanosoma cruzi
caggaccgtgatgtcatatcgcacgcggaggaagccgagcgcagtgctgtcctggcagcagtgcacagaggtgcgacggccattgctggtgcagaggccttgagtcggagagagcag

SEQ ID NO: 27 - DNA, 81 bp, Trypanosoma cruzi
atgcccctcccgttgcgcacggtgctggtgccttccgtgccacgcagtaacaccttcgccttctcctccacggcactgatc

SEQ ID NO: 28 - DNA, 81 bp, Trypanosoma cruzi
gtggccgtgcaaacggatgcactgcgtccccatggccctcccagggagcggacgacacaaaccgtgagggaattggaggag

SEQ ID NO: 29 - DNA, 435 bp, Trypanosoma cruzi
ggtgtgttctctctcgagcaatggagggactatgaaggaaaggatacggtcactccacttgcaaggagaaatctaaacagagtactcactcaattactgagagaagaaaggcgggaggcagaagaaagggcagtgcgggagggacatgtaggatttgccctaactactacgatcagggatgtgctatttagaggaagagttcgcgtcaaggacatgaagctgaatgattttcttatgatggaattggaaggcagaggcattttgcgcgccaatcggaatgtcttactgagggtttttttcagtgatcccacgagtcatatccgcgatgcgggagtattgaacgaaatacgggcatcaggtgcttatttgaggatggagatggctgtacgggaggaaatgggtttggaagaagttgcacgcagtctttgcgagaat

SEQ ID NO: 30 - DNA, 36 bp, Trypanosoma cruzi
gaggaagaggcagcccgaaggatgcatgaggtggcc

SEQ ID NO: 31 - DNA, 42 bp, Trypanosoma cruzi
gagaggctggcggatgagcttgagcagaaggcagcggagaat

SEQ ID NO: 32 - DNA, 30 bp, Trypanosoma cruzi
ccaccaagggctgcacctgctgcaccggaa

SEQ ID NO: 33 - DNA, 195 bp, Trypanosoma cruzi
aaacagttggcggcgagggaggcgccgagccgcatcaatgcgcaggaggagtacgacgcgctgaagagttttgtggataactctctgaagccgttgatatcgcgcctgaagcgcacgaacgaggagaaggaacttgacctgcgggcgaacgaggagcgcatccggcagctgctgcatgacaaggagcagcttcag

SEQ ID NO: 34 - DNA, 69 bp, Trypanosoma cruzi
cctgcgctctattctgctgaatatcgggatgccatctcgccgatgctccagtcgtgtgatgctctgcct

SEQ ID NO: 35 - DNA, 165 bp, Trypanosoma cruzi
ggcaagctgggtaacgtggacgcgcagttggagaagtatcgtggccatgaggaggagttcattgctgcactggagcagaagtatggtcccgagccatcagttgcggagagtgcctcaccagactaccgcagccgtgttgtgtccatctatgagcgctatgcccct

SEQ ID NO: 36 - DNA, 477 bp, Trypanosoma cruzi
gaggctgcacgtaagcgggctgaggaagaggcagcacgtaagcaggcggaggaagaggccgcacgcaagcaggctgaagaggccgcacgtaaacaggctgaggaagaggccgcacgcaagcaggcggaggaggaggccgcacgtaagcaggcggaggaagaggcagcacgtaagcaggcggaggaagaggccgcacgcaagcaggctgaggaagaggctgcacgtaagcaggctgaagaggaggccgcacgcaagcaggctgaggaagaggctgcacgcaagcgggctgaggaggctgcacgcaagaaggcacgtgaagaagctgagcgaaaaagggccgaagacgaggccgcacggaaaagagctcgaagagaagcgcgggaaagggcaaaggaaattgtgaagcagaggcgagctaaggaagaggccgcacgtaaacaggcggaggaggaggccgcacgcaagcaggctgaagag

SEQ ID NO: 37 - DNA, 60 bp, Trypanosoma cruzi
gatgagcagccagcaaacgtcactgcacggcggggcggcgttgccatgacatttgggaac

SEQ ID NO: 38 - DNA, 216 bp, Trypanosoma cruzi
accgaagatgtccggctgcgccttgagcgctttatgcggaagtacaatcctgggaagcttggcacaattgacggcattttgaagacatacaggggaagggaggagcagatgtttgaggcactggtgaggaagtacggcccggagccttcacgggatgagttgcctgacgctgcttctgccaaaaccgcccaatcctcctctgtacacactgaagaa

SEQ ID NO: 39 - DNA, 42 bp, Trypanosoma cruzi
agcctccctctgcgcaagttcctctgcgagcttctcattgtc

SEQ ID NO: 40 - DNA, 120 bp, Trypanosoma cruzi
ctctcgtcacaggaggtccaggctgtggtggatgcaaggcgtaccacgccaaggaatgacgagtacgacgatgactatgagtccgaaacacccgcttccgctaagaagcggcccgcggtg

SEQ ID NO: 41 - DNA, 162 bp, Trypanosoma cruzi
gagcggcagcgtgcggaggacgcggagcgctgtgcgcacgacactgctggcacagtggagcagcgccaccgtgagcagctggccacgctggaggccgccctggagcagcagcgggcacagcatgccagcgaggtggacgatcttcgcgtggcactggagcgc

SEQ ID NO: 42 - DNA, 129 bp, Trypanosoma cruzi
gtggccacactgacgcgggaggtgcagcggctggagcgggagctggaggaggctcggcaggagcaggagcgagccgttggtgccacggaggccatgcagcgcgacgccgcacgggagagagacgaggcc

SEQ ID NO: 43 - DNA, 69 bp, Trypanosoma cruzi
atgggcggtaatggtgcggtacccatgggcggaatggagggtgtcagaggtgcgggtccctacggaggc

SEQ ID NO: 44 - DNA, 435 bp, Trypanosoma cruzi
tgcatccgacgagcctcccgttgcagtcaagcagcgtgggcgaccgagaaggacgacgccgacggtgcccgttgcatccgacgagcctttcgatgcatccgacgagcctcccgttgcagtcaagcagcgtgggcgaccgaggacgacgccgacgatgcccgttgcatccgacgagcctttcgatgcatccgacgagcctcccgttgcagtcaagcagcgtgggcggccgagaaggacgacgccgacggtgcccgttgcatccgacgagtctcccgttgcagtcaagcagcgtgggcgaccgaggacgacgccgacgatgcccgttgcatccgacgagcctttcgatgcatccgacgagcctcccgttgcagtcaagcagcgcgggcgaccgaggacgacgccgacggtgcccgttgcatccgacgagcctttcga

SEQ ID NO: 45 - DNA, 117 bp, Trypanosoma cruzi
gagcgcgatgagctgcaggagcaccttgccgccacgagcgatgatctgggtaggcagctgcgtgctgtggagcaggcgaagacggaggtggagcagagcctggaggcaatgacgtcg

SEQ ID NO: 46 - DNA, 216 bp, Trypanosoma cruzi
atcacaacgaaggcgccaacgacgatcacaacaatggagccaaccacgatcaccacgaaggcaccaacgacgatcacaacaatggagccaaccacgatcaccacgaaggcgccaacgacgatcacaacaatggagccaaccacgatcaccacgaaggcgccaacgacgatcacaacaatggagccaacgacgatcaccacgaaggcaccaacgacg

SEQ ID NO: 47 - DNA, 18 bp, Trypanosoma cruzi
atgggggacgataacctt

SEQ ID NO: 48 - DNA, 231 bp, Trypanosoma cruzi
atgtcttcctaccgggaccgactggtggctttctacgagaagtatgcaccccgcaaggtggggcaggtggatgcacagctggagaagtacgccggacgtgaggaggacttttttgctgcgcttgtgcagaagtacggtcccgagccggggaatgctgccggtacgcctgcagccagcagaggtgccagccccgctttcagtgaatccgccacacccaccacaatggacaga

SEQ ID NO: 49 - DNA, 30 bp, Trypanosoma cruzi
gaggaggaagcggagaagaggcatcaagcg

SEQ ID NO: 50 - DNA, 114 bp, Trypanosoma cruzi
gagggaggccgcagcatggagtcgttgcctcagagtgttactgtggttcctcgtagtgaacgtcccatgagcaggaagtcgtttgatgtctcattggtgcacgagaacgagatg

SEQ ID NO: 51 - DNA, 60 bp, Trypanosoma cruzi
ggcggtcaacgtggtggcggcagacgtgactacggtgatcagggcaaccagggcgactat

SEQ ID NO: 52 - DNA, 18 bp, Trypanosoma cruzi
catgtgtatggctgtatg

SEQ ID NO: 53 - DNA, 72 bp, Trypanosoma cruzi
tccgacgagcctcccgtcgcagtcaagcagcgtgggcgaccgagaaggacgatgctgacggtgcccgatgca

SEQ ID NO: 54 - DNA, 123 bp, Trypanosoma cruzi
ctgcccaagaagaagggacgcccaaagaagctgaacgaggcagaggatgcggtggccgaaatgacattaatggaggaacaggaggagatgtcagcagccgtcacggaggcggaggaggcgcca

SEQ ID NO: 55 - DNA, 48 bp, Trypanosoma cruzi
aacactcaactgaatgccgaggtcaatgaggagacgccggtgagaagc

SEQ ID NO: 56 - DNA, 24 bp, Trypanosoma cruzi
gcgcctccaactgcgagtgtgcct

SEQ ID NO: 57 - DNA, 249 bp, Trypanosoma cruzi
tcggctggagcatacaagccacccacagacaggccatcggctggtgcgtacaagccacccacagacaggccatcggccggtgcgtacaagccacccacagacaggccatcggctggtgcgtacaagccacccacggaaaagccgtctatgaatgcagaggtgccatcaactaacaaaactgagggaacaccgatttcgcccacggacaagtcttcggctgaagcatacaagccacccacagacaggcca

SEQ ID NO: 58 - DNA, 72 bp, Trypanosoma cruzi
ggggaagacagcgattcgtcgggagcagccgacactgactccgcgaagggcaaagcaaccggcagttctgca

SEQ ID NO: 59 - DNA, 102 bp, Trypanosoma cruzi
gccgttccggacagctcctccgacgatgatgatgcgcccgtgcggaagccagcacaaaaggccaaggcgtcacccgcaatgaggccagcaccgaagaaggcc

SEQ ID NO: 60 - DNA, 396 bp, Trypanosoma cruzi
ggctctggcggctttgtgaggagcggtccaccggccgtgattcccgaggatgatggttttgcaccgaatgccggaagcgatgatgaaggcccaaagaagccttcaatcgcctcggcgccccccaagccattctctctgggcactgggtcactgccagcaaaacccgcacctggcacaggccccaattctgcgcccgttgccccgtcgaaccctttcagcttcggtaacagcagtggcggtgcggctccagctgacaagcccgctgccccgtcgaaccctttcagcttcggtaacaacagtggcggtgcggctccagctgacaagcccgctgcaccgtcgaattcctttggtgtcgttgtcagtaaggggttgactgtgcagggcgggaactctgcg

SEQ ID NO: 61 - DNA, 78 bp, Trypanosoma cruzi
tccgacagcgacgacgagccggtgcgcaagcccagcaagccatcacccaaggctgctcccaaaaaggccatggccgac

SEQ ID NO: 62 - DNA, 60 bp, Trypanosoma cruzi
caggcgtacaagggcgatgcggattccgaccaggcgtacatgaaacgcactgatatgggc

SEQ ID NO: 63 - DNA, 348 bp, Trypanosoma cruzi
tcacaaccagcaacgccgacgtcatccgtgacgacggagggagttgcatcttccccagcggatgcagggcctgaggactcccaggaagatggcgagaggctggcagaggagcttgtgcagctggtccagcgctgtggtcgtggtgccctggcgcgtcgcagcatgttttccgtgctctctgacaccccgcgatggaaggcggccgtggatatacagcgtgtgtggcgcggctactgttcgcgacagctcgtggaggtgtactatgagttccctgcggaggaagagaggacggaaggcgacagaagcgaaaaggggggaaacatggaaaatgtagaggagtttgtcaat

SEQ ID NO: 64 - DNA, 75 bp, Trypanosoma cruzi
agtcctgttgagcgtgatgcccgcgacaactctcgtgctgcccgtgatcgcaggaatcgtgaggatgaaccccgc

SEQ ID NO: 65 - DNA, 60 bp, Trypanosoma cruzi
gcatgcagcgtcaaccatcgtttggcggtcgtggtggcatgcccgttcagggacaaggag

SEQ ID NO: 66 - DNA, 255 bp, Trypanosoma cruzi
gaggaggctgcgaagcgcctggaggcggagctggaggaacgcacgaacgacctccaagagcgtgctgctgctgccgaggatgccgctcgccgtcgctgtgccgctgcaagggagaaggaggaggctgcgaagcgcctggaggcggagctggaggtgcgcacgaacgacctccaagagcgtgcaaacgacctccaagagcgtgctgctgctgccgaggatgccgctcgccgtcgctgtgccgctgcaagggagaag

SEQ ID NO: 67 - DNA, 222 bp, Trypanosoma cruzi
gagtacgaggcagttgcggcagccaaggcgaaggcggaggcggagcgcgacgacgcgcggcagaaattgcgtggggcggaggaaggactggagtccttccgccggcaggcagagtcgcgtcgagcccaaattgcaggactgcagtcggcggcgagctccacgagacccacaccgtccagcacgcggaatgcggcgcccccgctctacactgtcaccgccgag

SEQ ID NO: 68 - DNA, 102 bp, Trypanosoma cruzi
gggacggacgtccagcagaagtcgtctcttggtggcagcagtggggtgccggtgcctgttcctcaggcgacccagccggtagtgggaggcacatcgacggcg

SEQ ID NO: 69 - DNA, 87 bp, Trypanosoma cruzi
atgcagcacatgaattgctccatgtgcatgaggaatatggagaaccaccacaagaacagttctcaaatggcaggcatgtcgggtgca

SEQ ID NO: 70 - DNA, 159 bp, Trypanosoma cruzi
gagggccagatcgagcagctggaagtcgatgtggcggagcgtgaccagaagctcgaggagatgatggctgcgcagaaggaccttgaggaacgctatgcaagcgacgcccatgctgccgaggggaagcaggcagaaatgcagggccagatcgagcagctg

SEQ ID NO: 71 - DNA, 63 bp, Trypanosoma cruzi
ggctaccccgaagagaaagaggactcaaggagggaacggtccggaagggagggaagggagcga

SEQ ID NO: 72 - DNA, 297 bp, Trypanosoma cruzi
ggtcctgctggtcccggggatggtgctgctgctttttccggtcgtggtgctgctattgctgatgctgctattcctggtgctgctggtcctgttcgtggtgttgatgctcctattcctggtcctgctgttcccggtgatggtcctgctttttccggtcttggtgatgctggtcccggtcatgctgctgctattccaggtgccgctgttcccggtactgctgatcctgctgctattcctggttctgctgttctcggtcatggtgctattcctggttccatttctgatgctgctattcct

SEQ ID NO: 73 - DNA, 141 bp, Trypanosoma cruzi
aaggcagaagaggagccattgcaagaacaggggaagacggagcactcagaaactgaattcattcgcgaggagggggagcggctaaagagactttcggctgcaatctgctcatgggaggaagaaaagctacgggaattggca

SEQ ID NO: 74 - DNA, 99 bp, Trypanosoma cruzi
ccggaggctgccgccgagtttgactacagggagctcggtgaagccgaagccggccttgccagtgaacacaaggaagaagtgcccgaggaggtgaatgct

SEQ ID NO: 75 - DNA, 102 bp, Trypanosoma cruzi
caaacttcgacgccagttcccgcggccacatccgttgtgcagccaacgccgagcaccttgtcggaagctgcaggagctgcaatgacttcggcagggacagca

SEQ ID NO: 76 - DNA, 48 bp, Trypanosoma cruzi
atggcacagccatctccacagcagcaacaggcgatgctgtcgacctcc

SEQ ID NO: 77 - DNA, 60 bp, Trypanosoma cruzi
aagaggcgtgctgagcaggaagagatggcgaggaggcgtgctgagcaggaagaggaggcg

SEQ ID NO: 78 - DNA, 48 bp, Trypanosoma cruzi
acggtgacgcctagccggacggctgtgcccgataggacgacttccagg

SEQ ID NO: 79 - DNA, 33 bp, Trypanosoma cruzi
caaggaagctttgctggagggggaatgccgcgc

SEQ ID NO: 80 - DNA, 48 bp, Trypanosoma cruzi
ccaccgccaccgccgccaccaccgggcgctggagcaaagtctggactt

SEQ ID NO: 81 - DNA, 219 bp, Trypanosoma cruzi
ccggaaatttctccaagggccaaggccatggagaacggtgctccattttatgagcgtctttatcaagtaaaagccgatgtggaaccaagccgcgaaggaaaatcccttctgcaatcgccgcctatatcgccccgtcgaccaacacgacccattcaattgtctcctcggcttctggagcgtcctgcaccaccggcagaaacgcttccgtactcgttccat

SEQ ID NO: 82 - DNA, 30 bp, Trypanosoma cruzi
ggtggcttcgggacagcggccaacacagct

SEQ ID NO: 83 - DNA, 48 bp, Trypanosoma cruzi
aagccgcagcaggcgcagagccctcagaaccgacccagcaatcagcag

SEQ ID NO: 84 - DNA, 198 bp, Trypanosoma cruzi
agtgagttacgcactccacatgaaagccaacacggtcgcattctgcccaaccggagccatccaacagccagtcaacgtgaacctccgacaccgcatgcgtctcttaatggctctaatttcatgcaggggtcgcaaaatgccaaggggcttcccgtacgcagcaatcccacaaacagtcgccgggagcttgggacacca

SEQ ID NO: 85 - DNA, 39 bp, Trypanosoma cruzi
ggtgctcccgttcccggtaatggccctgctgctattcct

SEQ ID NO: 86 - DNA, 51 bp, Trypanosoma cruzi
gcacagggacagagtgcacaacaaagtcctcagaattatgcttcactgcag

SEQ ID NO: 87 - DNA, 33 bp, Trypanosoma cruzi
ggtggcttccgtggaggacgcggtggcgatcgt

SEQ ID NO: 88 - DNA, 126 bp, Trypanosoma cruzi
ctctgcgaaacaccgagccgtgcggggaatgaatccaatgcgctctgcgaaacaccgagccgtgcggggaatgaatccaatgcgctctgcgaaacaccgagccgtgcggaggatgagtctaatgcg

SEQ ID NO: 89 - DNA, 39 bp, Trypanosoma cruzi
ggcatgggaggttccatgtatggtatgggcggtcctatg

SEQ ID NO: 90 - DNA, 168 bp, Trypanosoma cruzi
acagcagcagcggaagcagaagcaaaagcagcagcagcatcagaagcagcaaaagcagcagccacacaagcaacagcaacagcggaagcagcaacaaaggcaaaagcagcagcagaaaaagcaaaagaagaagcagcaacagcggcagcagcagaagcagtaacagca

SEQ ID NO: 91 - DNA, 21 bp, Trypanosoma cruzi
ggtgctggtggattccctggc

SEQ ID NO: 92 - DNA, 90 bp, Trypanosoma cruzi
gagcctgcggaggatgctgagagggaggccgcgccgccgctgcacagcacggaggacattgtccctgcggacacggagagggagctgagt

SEQ ID NO: 93 - DNA, 36 bp, Trypanosoma cruzi
gccaaaagtacgtcctcaactcccgttggcagcggt

SEQ ID NO: 94 - DNA, 63 bp, Trypanosoma cruzi
cctcagccaggatacggggcgcctcagccaggatacggaccgcctcagccaggatacggggcg

SEQ ID NO: 95 - DNA, 147 bp, Trypanosoma cruzi
ccatccagccaaaacccgacacaggaggcttacaggccgatgccatccagttccaagtctaaacctgacgtctacaacccgacacaggaggcttacaggtcgatgccatccagttccaagtctaaacctgacgtctacaaccagact

SEQ ID NO: 96 - DNA, 33 bp, Trypanosoma cruzi
acaaccacgacgacaaagccaccaacgacgact

SEQ ID NO: 97 - protein, 65 aa, Trypanosoma cruzi
LFRIMNGGGQRHSLSMSVCVFTHWTPNNPELSAAWRNFARSCPQGCCTRRIVFERLLHHTLWSKF

SEQ ID NO: 98 - protein, 42 aa, Trypanosoma cruzi
ESVDAVAAMERQLQERDDALAALKDRLEEYSREKSALESRTS

SEQ ID NO: 99 - protein, 14 aa, Trypanosoma cruzi
DAVAENAQLQKERD

SEQ ID NO: 100 - protein, 160 aa, Trypanosoma cruzi
SASTAWVWITTRRSRWRPPCLRTRRRMRLLSDSASTAWVWITTRRSRWRPPCLRTRRRMRLLSDSASTAWVWITTRRSRWRPPCLRTRRRMRLLSDSASTAWVWSTTRRSRWRPPCLRTRRRMRLLSDSASTAWVWITTRRSRWRPPCLRTRRRMRLLSD

SEQ ID NO: 101 - protein, 15 aa, Trypanosoma cruzi
SGVGSFMSEGSSKER

SEQ ID NO: 102 - protein, 42 aa, Trypanosoma cruzi
LAQREADNEKLAEELAQREADNEKLAEELAQREADNEKLAED

SEQ ID NO: 103 - protein, 24 aa, Trypanosoma cruzi
LQRQNEELQSQLKESRRGEEKLDA

SEQ ID NO: 104 - protein, 35 aa, Trypanosoma cruzi
ENEELRAEHEHKTRGLQEVSEQAEDLQRQLEELRA

SEQ ID NO: 105 - protein, 42 aa, Trypanosoma cruzi
PETASVPEEHRGSRKVSGAVPEGEEYPTEAERPVATDEDGHA

SEQ ID NO: 106 - protein, 10 aa, Trypanosoma cruzi
ARRKAAEEEA

SEQ ID NO: 107 - protein, 106 aa, Trypanosoma cruzi
WQEQERERRAKEAEEAKRQLEQELKAQQEAREAEERRRVELAKQQREESKARKEELQRKQAEERRKKKEELQAETERLLAEARSAEEGEKKALAEKVRTGKEEGAR

SEQ ID NO: 108 - protein, 9 aa, Trypanosoma cruzi
EAEARRLAE

SEQ ID NO: 109 - protein, 39 aa, Trypanosoma cruzi
QLDEAVQQREEVQRELERTIEELAALREQSGVDTVNLRA

SEQ ID NO: 110 - protein, 39 aa, Trypanosoma cruzi
MATDELTTEARPLDVTVGSALLSDSAAVTAPADRDATAL

SEQ ID NO: 111 - protein, 20 aa, Trypanosoma cruzi
TTQPLGGTGVTTATTQPFSA

SEQ ID NO: 112 - protein, 14 aa, Trypanosoma cruzi
GWRMSLSRRQRRMR

SEQ ID NO: 113 - protein, 8 aa, Trypanosoma cruzi
EAEKRAQR

SEQ ID NO: 114 - protein, 30 aa, Trypanosoma cruzi
EDVQELVTPAEDVQELVAPAEDIQELVAPA

SEQ ID NO: 115 - protein, 14 aa, Trypanosoma cruzi
RRGMTPWRRMPSCR

SEQ ID NO: 116 - protein, 124 aa, Trypanosoma cruzi
TTSKSVLLLPRMPLAVAVPLQGRRRRLRSAWRRSWRNARTTSKSVLLLPRMPLAVAVPLQGRRRRLRSAWRRSWRCARTTSKSVQTISKSVLLLPRMPLAVAVPLQGRRRRLRSAWRRSWRSAR

SEQ ID NO: 117 - protein, 40 aa, Trypanosoma cruzi
DSENPMSGAAASSILVKGAGNSSSTNTMRNMRNASVAVQE

SEQ ID NO: 118 - protein, 28 aa, Trypanosoma cruzi
VTAEREELAENLRATEDAKAEVERNLES

SEQ ID NO: 119 - protein, 65 aa, Trypanosoma cruzi
AARGQLVGEEREGTCGLHRDAVDSEERAVRRCLERGEAAAVDELGEEYGSATHERVVEALAAEED

SEQ ID NO: 120 - protein, 14 aa, Trypanosoma cruzi
GFGSATHTSAPAAG

SEQ ID NO: 121 - protein, 18 aa, Trypanosoma cruzi
SPTLTETEKGTGTPAHTA

SEQ ID NO: 122 - protein, 39 aa, Trypanosoma cruzi
QDRDVISHAEEAERSAVLAAVHRGATAIAGAEALSRREQ

SEQ ID NO: 123 - protein, 27 aa, Trypanosoma cruzi
MPLPLRTVLVPSVPRSNTFAFSSTALI

SEQ ID NO: 124 - protein, 27 aa, Trypanosoma cruzi
VAVQTDALRPHGPPRERTTQTVRELEE

SEQ ID NO: 125 - protein, 145 aa, Trypanosoma cruzi
GVFSLEQWRDYEGKDTVTPLARRNLNRVLTQLLREERREAEERAVREGHVGFALTTTIRDVLFRGRVRVKDMKLNDFLMMELEGRGILRANRNVLLRVFFSDPTSHIRDAGVLNEIRASGAYLRMEMAVREEMGLEEVARSLCEN

SEQ ID NO: 126 - protein, 12 aa, Trypanosoma cruzi
EEEAARRMHEVA

SEQ ID NO: 127 - protein, 14 aa, Trypanosoma cruzi
ERLADELEQKAAEN

SEQ ID NO: 128 - protein, 10 aa, Trypanosoma cruzi
PPRAAPAAPE

SEQ ID NO: 129 - protein, 65 aa, Trypanosoma cruzi
KQLAAREAPSRINAQEEYDALKSFVDNSLKPLISRLKRTNEEKELDLRANEERIRQLLHDKEQLQ

SEQ ID NO: 130 - protein, 23 aa, Trypanosoma cruzi
PALYSAEYRDAISPMLQSCDALP

SEQ ID NO: 131 - protein, 55 aa, Trypanosoma cruzi
GKLGNVDAQLEKYRGHEEEFIAALEQKYGPEPSVAESASPDYRSRVVSIYERYAP

SEQ ID NO: 132 - protein, 159 aa, Trypanosoma cruzi
EAARKRAEEEAARKQAEEEAARKQAEEAARKQAEEEAARKQAEEEAARKQAEEEAARKQAEEEAARKQAEEEAARKQAEEEAARKQAEEEAARKRAEEAARKKAREEAERKRAEDEAARKRARREARERAKEIVKQRRAKEEAARKQAEEEAARKQAEE

SEQ ID NO: 133 - protein, 20 aa, Trypanosoma cruzi
DEQPANVTARRGGVAMTFGN

SEQ ID NO: 134 - protein, 72 aa, Trypanosoma cruzi
TEDVRLRLERFMRKYNPGKLGTIDGILKTYRGREEQMFEALVRKYGPEPSRDELPDAASAKTAQSSSVHTEE

SEQ ID NO: 135 - protein, 14 aa, Trypanosoma cruzi
SLPLRKFLCELLIV

SEQ ID NO: 136 - protein, 40 aa, Trypanosoma cruzi
LSSQEVQAVVDARRTTPRNDEYDDDYESETPASAKKRPAV

SEQ ID NO: 137 - protein, 54 aa, Trypanosoma cruzi
ERQRAEDAERCAHDTAGTVEQRHREQLATLEAALEQQRAQHASEVDDLRVALER

SEQ ID NO: 138 - protein, 43 aa, Trypanosoma cruzi
VATLTREVQRLERELEEARQEQERAVGATEAMQRDAARERDEA

SEQ ID NO: 139 - protein, 23 aa, Trypanosoma cruzi
MGGNGAVPMGGMEGVRGAGPYGG

SEQ ID NO: 140 - protein, 145 aa, Trypanosoma cruzi
CIRRASRCSQAAWATEKDDADGARCIRRAFRCIRRASRCSQAAWATEDDADDARCIRRAFRCIRRASRCSQAAWAAEKDDADGARCIRRVSRCSQAAWATEDDADDARCIRRAFRCIRRASRCSQAARATEDDADGARCIRRAFR

SEQ ID NO: 141 - protein, 39 aa, Trypanosoma cruzi
ERDELQEHLAATSDDLGRQLRAVEQAKTEVEQSLEAMTS

SEQ ID NO: 142 - protein, 72 aa, Trypanosoma cruzi
ITTKAPTTITTMEPTTITTKAPTTITTMEPTTITTKAPTTITTMEPTTITTKAPTTITTMEPTTITTKAPTT

SEQ ID NO: 143 - protein, 6 aa, Trypanosoma cruzi
MGDDNL

SEQ ID NO: 144 - protein, 77 aa, Trypanosoma cruzi
MSSYRDRLVAFYEKYAPRKVGQVDAQLEKYAGREEDFFAALVQKYGPEPGNAAGTPAASRGASPAFSESATPTTMDR

SEQ ID NO: 145 - protein, 10 aa, Trypanosoma cruzi
EEEAEKRHQA

SEQ ID NO: 146 - protein, 38 aa, Trypanosoma cruzi
EGGRSMESLPQSVTVVPRSERPMSRKSFDVSLVHENEM

SEQ ID NO: 147 - protein, 20 aa, Trypanosoma cruzi
GGQRGGGRRDYGDQGNQGDY

SEQ ID NO: 148 - protein, 6 aa, Trypanosoma cruzi
HVYGCM

SEQ ID NO: 149 - protein, 24 aa, Trypanosoma cruzi
SDEPPVAVKQRGRPRRTMLTVPDA

SEQ ID NO: 150 - protein, 41 aa, Trypanosoma cruzi
LPKKKGRPKKLNEAEDAVAEMTLMEEQEEMSAAVTEAEEAP

SEQ ID NO: 151 - protein, 16 aa, Trypanosoma cruzi
NTQLNAEVNEETPVRS

SEQ ID NO: 152 - protein, 8 aa, Trypanosoma cruzi
APPTASVP

SEQ ID NO: 153 - protein, 83 aa, Trypanosoma cruzi
SAGAYKPPTDRPSAGAYKPPTDRPSAGAYKPPTDRPSAGAYKPPTEKPSMNAEVPSTNKTEGTPISPTDKSSAEAYKPPTDRP

SEQ ID NO: 154 - protein, 24 aa, Trypanosoma cruzi
GEDSDSSGAADTDSAKGKATGSSA

SEQ ID NO: 155 - protein, 34 aa, Trypanosoma cruzi
AVPDSSSDDDDAPVRKPAQKAKASPAMRPAPKKA

SEQ ID NO: 156 - protein, 132 aa, Trypanosoma cruzi
GSGGFVRSGPPAVIPEDDGFAPNAGSDDEGPKKPSIASAPPKPFSLGTGSLPAKPAPGTGPNSAPVAPSNPFSFGNSSGGAAPADKPAAPSNPFSFGNNSGGAAPADKPAAPSNSFGVVVSKGLTVQGGNSA

SEQ ID NO: 157 - protein, 26 aa, Trypanosoma cruzi
SDSDDEPVRKPSKPSPKAAPKKAMAD

SEQ ID NO: 158 - protein, 20 aa, Trypanosoma cruzi
QAYKGDADSDQAYMKRTDMG

SEQ ID NO: 159 - protein, 116 aa, Trypanosoma cruzi
SQPATPTSSVTTEGVASSPADAGPEDSQEDGERLAEELVQLVQRCGRGALARRSMFSVLSDTPRWKAAVDIQRVWRGYCSRQLVEVYYEFPAEEERTEGDRSEKGGNMENVEEFVN

SEQ ID NO: 160 - protein, 25 aa, Trypanosoma cruzi
SPVERDARDNSRAARDRRNREDEPR

SEQ ID NO: 161 - protein, 20 aa, Trypanosoma cruzi
ACSVNHRLAVVVACPFRDKE

SEQ ID NO: 162 - protein, 85 aa, Trypanosoma cruzi
EEAAKRLEAELEERTNDLQERAAAAEDAARRRCAAAREKEEAAKRLEAELEVRTNDLQERANDLQERAAAAEDAARRRCAAAREK

SEQ ID NO: 163 - protein, 74 aa, Trypanosoma cruzi
EYEAVAAAKAKAEAERDDARQKLRGAEEGLESFRRQAESRRAQIAGLQSAASSTRPTPSSTRNAAPPLYTVTAE

SEQ ID NO: 164 - protein, 34 aa, Trypanosoma cruzi
GTDVQQKSSLGGSSGVPVPVPQATQPVVGGTSTA

SEQ ID NO: 165 - protein, 29 aa, Trypanosoma cruzi
MQHMNCSMCMRNMENHHKNSSQMAGMSGA

SEQ ID NO: 166 - protein, 53 aa, Trypanosoma cruzi
EGQIEQLEVDVAERDQKLEEMMAAQKDLEERYASDAHAAEGKQAEMQGQIEQL

SEQ ID NO: 167 - protein, 21 aa, Trypanosoma cruzi
GYPEEKEDSRRERSGREGRER

SEQ ID NO: 168 - protein, 99 aa, Trypanosoma cruzi
GPAGPGDGAAAFSGRGAAIADAAIPGAAGPVRGVDAPIPGPAVPGDGPAFSGLGDAGPGHAAAIPGAAVPGTADPAAIPGSAVLGHGAIPGSISDAAIP

SEQ ID NO: 169 - protein, 47 aa, Trypanosoma cruzi
KAEEEPLQEQGKTEHSETEFIREEGERLKRLSAAICSWEEEKLRELA

SEQ ID NO: 170 - protein, 33 aa, Trypanosoma cruzi
PEAAAEFDYRELGEAEAGLASEHKEEVPEEVNA

SEQ ID NO: 171 - protein, 34 aa, Trypanosoma cruzi
QTSTPVPAATSVVQPTPSTLSEAAGAAMTSAGTA

SEQ ID NO: 172 - protein, 16 aa, Trypanosoma cruzi
MAQPSPQQQQAMLSTS

SEQ ID NO: 173 - protein, 20 aa, Trypanosoma cruzi
KRRAEQEEMARRRAEQEEEA

SEQ ID NO: 174 - protein, 16 aa, Trypanosoma cruzi
TVTPSRTAVPDRTTSR

SEQ ID NO: 175 - protein, 11 aa, Trypanosoma cruzi
QGSFAGGGMPR

SEQ ID NO: 176 - protein, 16 aa, Trypanosoma cruzi
PPPPPPPPGAGAKSGL

SEQ ID NO: 177 - protein, 73 aa, Trypanosoma cruzi
PEISPRAKAMENGAPFYERLYQVKADVEPSREGKSLLQSPPISPRRPTRPIQLSPRLLERPAPPAETLPYSFH

SEQ ID NO: 178 - protein, 10 aa, Trypanosoma cruzi
GGFGTAANTA

SEQ ID NO: 179 - protein, 16 aa, Trypanosoma cruzi
KPQQAQSPQNRPSNQQ

SEQ ID NO: 180 - protein, 66 aa, Trypanosoma cruzi
SELRTPHESQHGRILPNRSHPTASQREPPTPHASLNGSNFMQGSQNAKGLPVRSNPTNSRRELGTP

SEQ ID NO: 181 - protein, 13 aa, Trypanosoma cruzi
GAPVPGNGPAAIP

SEQ ID NO: 182 - protein, 17 aa, Trypanosoma cruzi
AQGQSAQQSPQNYASLQ

SEQ ID NO: 183 - protein, 11 aa, Trypanosoma cruzi
GGFRGGRGGDR

SEQ ID NO: 184 - protein, 42 aa, Trypanosoma cruzi
LCETPSRAGNESNALCETPSRAGNESNALCETPSRAEDESNA

SEQ ID NO: 185 - protein, 13 aa, Trypanosoma cruzi
GMGGSMYGMGGPM

SEQ ID NO: 186 - protein, 56 aa, Trypanosoma cruzi
TAAAEAEAKAAAASEAAKAAATQATATAEAATKAKAAAEKAKEEAATAAAAEAVTA

SEQ ID NO: 187 - protein, 7 aa, Trypanosoma cruzi
GAGGFPG

SEQ ID NO: 188 - protein, 30 aa, Trypanosoma cruzi
EPAEDAEREAAPPLHSTEDIVPADTERELS

SEQ ID NO: 189 - protein, 12 aa, Trypanosoma cruzi
AKSTSSTPVGSG

SEQ ID NO: 190 - protein, 21 aa, Trypanosoma cruzi
PQPGYGAPQPGYGPPQPGYGA

SEQ ID NO: 191 - protein, 49 aa, Trypanosoma cruzi
PSSQNPTQEAYRPMPSSSKSKPDVYNPTQEAYRSMPSSSKSKPDVYNQT

SEQ ID NO: 192 - protein, 11 aa, Trypanosoma cruzi
TTTTTKPPTTT

SEQ ID NO: 193 - DNA, 30 bp, Trypanosoma cruzi
caattacatatgagcgcgagcaccgcctgg

SEQ ID NO: 194 - DNA, 34 bp, Trypanosoma cruzi
caattaaagcttctagtcgctcaacaaccgcatg

SEQ ID NO: 195 - DNA, 31 bp, Trypanosoma cruzi
caattacatatggagaacgaggagctgcgtg

SEQ ID NO: 196 - DNA, 33 bp, Trypanosoma cruzi
caattaaagcttctacgcacgaagctcctccag

SEQ ID NO: 197 - DNA, 30 bp, Trypanosoma cruzi
caattacatatgccggagacagcctcagtc

SEQ ID NO: 198 - DNA, 33 bp, Trypanosoma cruzi
caattaaagcttctacgcgtgaccgtcctcgtc

SEQ ID NO: 199 - DNA, 27 bp, Trypanosoma cruzi
caattacatatggcaacggacgagttg

SEQ ID NO: 200 - DNA, 34 bp, Trypanosoma cruzi
caattaaagcttctagagcgcagtcgcatccctg

(19) United States
(12) Patent Application Publication
(10) Pub. No.: US 2009/0325204 A1
(43) Pub. Date: Dec. 31, 2009

(54) COMPOUNDS AND METHODS FOR DIAGNOSIS AND TREATMENT OF CHAGAS DISEASE

(76) Inventors: Steven G. Reed, Bellevue, WA (US); Yasuyuki Goto, Seattle, WA (US)

Correspondence Address: DYLAN O. ADAMS;Graybeal Jackson LLP, 155 - 108th Avenue N.E., Suite 350, Bellevue, WA, 98004-5901, US

(21) Appl. No.: 12/494,206

(22) Filed: Jun. 29, 2009

Related U.S. Application Data
(60) Provisional application No. 61/076,511, filed on Jun. 27, 2008.

(51) Int. Cl. G01N 33/53; C12N 1/21; C12N 15/63; C07K 2/00; C07H 21/04

(52) U.S. Cl. 435/7.92; 435/252.3; 435/320.1; 530/300; 536/23.4

(57) ABSTRACT

Compounds and methods are provided herein that provide for diagnosis and treatment of Chagas disease.

### RELATED REFERENCES

This application claims priority to U.S. Provisional Application 61/076,511 filed Jun. 27, 2008 entitled “COMPOUNDS AND METHODS FOR DIAGNOSIS AND TREATMENT OF CHAGAS DISEASE”. The foregoing application is hereby incorporated by reference in its entirety as if fully set forth herein.

### FIELD

This invention relates generally to infectious diseases, and more specifically, to compounds and methods for diagnosis and treatment of Chagas disease.

### BACKGROUND

American trypanosomiasis (Chagas disease) is a protozoan infection caused by the flagellate Trypanosoma (Schizotrypanum) cruzi, widespread in the Americas, and endemic to Central and South America. Chagas disease may be quickly fatal, especially in children, or it may be carried asymptomatically for decades. Chagas disease is characterized by a short-term acute phase, with very few clinical symptoms, and a long-term chronic phase, usually accompanied by severe gastrointestinal and/or cardiac complications which result in permanent physical disability or death. Between 10-30% of infected people eventually develop severe cardiac or digestive chronic involvement as late manifestations of Chagas disease. In the Americas, approximately 16-18 million people are estimated to be infected by the parasite and as many as 90 million individuals are at risk of infection (World Health Organization, 1991). It is estimated that the infection causes 50,000 deaths annually (Carlier, Yves M.D. eMedicine.com, 2003). These estimates do not include Mexico and Nicaragua, for which accurate public health data are not available.

Due to patterns of urbanization and immigration, Chagas disease is no longer a unique problem for Latin American countries. Estimates have suggested that approximately 300,000 infected individuals were living in the city of Sao Paulo and more than 200,000 in Rio de Janeiro and Buenos Aires. In addition, Chagasic patients with chronic and asymptomatic forms of the disease are immigrating northward to the United States and Canada, and even eastward to Europe. It has been estimated that around 500,000 infected individuals were already living in the United States, most of them having immigrated from Mexico and Central America (National Institutes of Health, 2006). Many of these immigrants are unaware that they have contracted Chagas disease and continue to donate infected blood. Controlling “transfusional” Chagas disease is therefore of paramount importance in preventing infection in the United States and Canada.

The disease is transmitted in many cases by Triatominae vectors, however blood transfusion is also an important form of transmission of the disease today. In Latin America, blood samples with antibodies associated with Chagas disease represent 1-4% of the total blood samples in major hemocenters.

The diagnosis of acute Chagas disease is typically not problematic because of the large number of parasites in the blood. In contrast, the chronic phase is diagnosed by serological methods because of the very small number or absence of circulating parasites. This has also restricted the use of polymerase chain reaction (PCR) with specific primers, as the final diagnostic test of Chagas disease, before a major epidemiologic survey of sera from chronic patients is carried out.

Presently, no optimal test is available for the diagnosis of chronic-stage Chagas disease. The most straightforward available method of excluding potentially infected donors from the blood pool is to ask questions about immigration and travel involving Central and South America. These geographic exclusions are somewhat insensitive and subject to the reliability of the potential donor. As a result, a large number of willing and healthy donors are inappropriately excluded, thus contributing to a blood donor shortage in Canada and the United States.

Serological tests for Chagas disease include the indirect fluorescent antibody (IFA) test and ELISA using whole cell antigen or recombinant antigens. Approved in December 2006, the ORTHO T. cruzi ELISA Test System (www.orthoclinical.com/chagas/elisaTestSystem.aspx) is the first such test approved by the FDA and is currently in use at Blood Banks in the United States for blood screening purposes.

However, there are deficiencies in these currently available diagnostic tests, especially as used in developing countries. The ORTHO T. cruzi ELISA Test System is not specific for T. cruzi infection. It detects antibodies in sera from about 75% of patients with leishmaniasis, caused by Leishmania parasites, which has overlapping endemicity with Chagas disease. This cross-reactivity can be caused by use of T. cruzi whole cell lysate as a source of the antigen used in the assay because a number of proteins are conserved between T. cruzi and Leishmania species. Visceral leishmaniasis is also a blood borne pathogen, and blood from these individuals must be eliminated from the supply. However, misdiagnosis may have serious consequences, particularly failure to provide adequate treatment to the infected donor.

In addition, there is documented cross reactivity of T. cruzi antigens with sera from other patient groups, including autoimmunity and syphilis. Aside from issues with specificity, serological tests used to diagnose T. cruzi infection are imperfect with sensitivity lacking in some regions including Peru and Brazil. Furthermore, there are currently no available rapid tests for Chagas disease. Compared with ELISA, which may take hours to perform, rapid tests such as an immuno-chromatographic test require only 5-10 min to get results and require a minimal level of training to perform and analyze. This enhanced throughput capability makes the rapid tests more user-friendly and feasible to use at local hospitals and even at field sites in developing countries.

However, rapid tests using crude lysates are less sensitive than ELISA, and defined antigens are required to provide the high epitope density needed to provide adequate sensitivity in a rapid test format. With proper selection, such antigens may also provide the specificity required for optimal test performance, something crude lysates cannot do. Despite significant improvements in T. cruzi diagnostics, current serology is achieved with the use of defined antigens, and no single antigen or combination has thus far been adequate for the development of efficient and cost effective rapid tests. The discovery of antigens with serological significance is an important factor for the development and improvement of diagnostic tests for T. cruzi infection.

### BRIEF DESCRIPTION OF THE DRAWINGS

The present invention will be described by way of exemplary embodiments but not limitations, illustrated in the accompanying drawings in which like references denote similar elements, and in which:

FIG. 1 is a flow diagram illustrating an antigenic polypeptide candidate selection routine in accordance with one embodiment.

FIG. 2 is a flow diagram illustrating an antigenic tandem repeat polypeptide candidate selection subroutine in accordance with one embodiment.

FIG. 3 is a flow diagram illustrating an antigenic tandem repeat polypeptide candidate homology screening subroutine in accordance with one embodiment.

FIG. 4 is a flow diagram illustrating an antigenic tandem repeat polypeptide candidate characteristic screening subroutine in accordance with one embodiment.

FIG. 5 is a flow diagram illustrating an antigenic tandem repeat polypeptide screening routine in accordance with one embodiment.

FIG. 6 is a table depicting the results of screening the genomes of various organisms for tandem repeat sequences.

FIG. 7 is a table depicting forty tandem repeat genes selected according to an embodiment.

FIG. 8 is a table depicting the nucleotide sequences and polypeptide sequences of ninety six tandem repeat genes selected according to another embodiment.

FIG. 9 depicts antibody responses of sera from visceral leishmaniasis patients, Chagas disease patents and healthy subjects to selected T. Cruzi tandem repeat proteins, which has been tested by ELISA.

FIG. 10a depicts the reactivity of T. cruzi antigen TcF in Ecuador and Brazil compared to a control. Sera from Ecuadorian (Ecu) or Brazilian (Bra) Chagas patients and Brazilian healthy endemic controls (Con) were examined compared to the reactivity to a diagnostic fusion antigen TcF by ELISA.

FIG. 10b depicts the reactivity of the combination of Tc6 (See SEQ ID NO: 14 and 110) and TcD compared to the reactivity of TcF alone in both healthy patients and patients having Chagas disease.

### DESCRIPTION

Illustrative embodiments presented herein include, but are not limited to, compounds and methods for diagnosis and treatment of Chagas disease.

Various aspects of the illustrative embodiments will be described using terms commonly employed by those skilled in the art to convey the substance of their work to others skilled in the art. However, it will be apparent to those skilled in the art that the embodiments described herein may be practiced with only some of the described aspects. For purposes of explanation, specific numbers, materials and configurations are set forth in order to provide a thorough understanding of the illustrative embodiments. However, it will be apparent to one skilled in the art that the embodiments described herein may be practiced without the specific details. In other instances, well-known features are omitted or simplified in order not to obscure the illustrative embodiments.

Further, various operations and/or communications will be described as multiple discrete operations and/or communications, in turn, in a manner that is most helpful in understanding the embodiments described herein; however, the order of description should not be construed as to imply that these operations and/or communications are necessarily order dependent. In particular, these operations and/or communications need not be performed in the order of presentation.

The phrase “in one embodiment” is used repeatedly. The phrase generally does not refer to the same embodiment; however, it may. The terms “comprising,” “having” and “including” are synonymous, unless the context dictates otherwise.

Unless defined otherwise, all technical and scientific terms used herein have the same meaning as commonly understood by those of ordinary skill in the art to which this invention pertains. The following references provide one of skill with a general definition of many of the terms used in this application: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). Exemplary methods and materials are described herein; however, various methods and materials similar or equivalent to those described herein may be used in the practice or testing of the present invention, and the exemplary methods and materials should not be construed to limit the scope or spirit of the present invention.

### Polypeptides According to Various Embodiments

Disclosed herein are exemplary polypeptides from Trypanosoma cruzi (“T. cruzi”); however, descriptions of these polypeptides should not be construed to limit the various embodiments. Various embodiments are directed to polypeptides from viruses, bacteria, pathogenic microbial agent, and other organisms.

Accordingly, some embodiments may be directed to polypeptides from various viruses including, but not limited to an immunodeficiency virus, Varicella zoster virus (“VZV”), cytomegalovirus (“CMV”), Colorado tick fever virus, dengue hemorrhagic fever virus, ebola hemorrhagic fever virus, hand, foot and mouth disease virus (“HFMD”), hepatitis virus, herpes simplex, herpes zoster, human papilloma virus (“HPV”), influenza virus, Lassa virus, Morbilliviurs, Marburgvirus, mononucleosis, Rubulavirus, norovirus, poliovirus, JC polyomavirus, Lyssavirus, Rubella virys, SARS coronavirus, and the like.

Additionally, some embodiments may be directed to polypeptides from various bacteria, including Bacillus anthracis, Meningitis causing bacteria, Clostridium botulinum, Brucella, Campylobacter jejuni, Bartonella, Vibrio cholerae, Corynebacterium diphtheriae, Salmonella enterica, Neisseria gonorrhoeae, Staphylococcus aureus, Legionella pneumophila, Mycobacterium leprae, Leptospira, Listeria monocytogenes, Borrelia burgdorferi, Burkholderia pseudomallei, Methicillin-resistant Staphylococcus aureus, Nocardia asteroides, Nocardia brasiliensis, Bordetella pertussis, Yersinia pestis, Streptococcus pneumoniae, Chlamydophila psittaci, Coxiella burnetii, Rickettsia ricketsii, Streptococcus pyogenes, Shigella, Treponema pallidum, Clostridium tetani, Chlamydia trachomatis, Mycobacterium tuberculosis, Francisella tularensis, Salmonella enterica, and the like.

Additionally, some embodiments may be directed to polypeptides from various organisms such as Trypanosoma, Entamoeba histolytica, Ascaris lumbricoides, Babesia, Clonorchis sinensis, Cryptosporidium, Taenia solium, Diphyllobothrium, Dracunculus medinensis, Echinococcus, Enterobius vermicularis, Fasciola hepatica, Fasciola gigantica, Wuchereria bancrofti, Brugia malayi, Brugia timori, Giardia lamblia, Gnathostoma spinigerum, Gnathostoma hispidum, Hymenolepis nana, Hymenolepis diminuta, Isospora belli, Leishmania, Plasmodium, Metagonimus yokogawai, Onchocerca volvulus, Schistosoma, Toxoplasma gondii, Trichinella spiralis, Trichuris trichiura, Trichomonas vaginalis, Trypanosoma brucei gambiense, Trypanosoma brucei rhodesiense, Aspergillus fumigatus, Blastomyces dermatitidis, Candida albicans, Coccidioides immitis, Coccidioides posadasii, Cryptococcus neoforman, Histoplasma capsulatum, and the like.

Polypeptides according to various exemplary embodiments described herein include, but are not limited to, polypeptides comprising immunogenic portions of Trypanosoma cruzi antigens comprising the sequences recited in SEQ ID NO: 97-192.

As used herein, the term “polypeptide” encompasses amino acid chains of any length, including full length proteins (e.g., antigens), wherein the amino acid residues are covalently linked as linear polymers by peptide bonds. Thus, a polypeptide comprising an immunogenic portion of one of the above antigens may consist entirely of the immunogenic portion, or may contain additional sequences. The additional sequences may be naturally occurring sequences such as sequences derived from the native T. cruzi antigen, or may be heterologous (e.g., derived from other sources including exogenous naturally occurring sequences and/or artificial sequences), and such sequences may (but need not) be immunogenic or antigenic.

An antigen “having” a particular recited sequence is an antigen that comprises a recited sequence, e.g., that contains, within its full length sequence, the recited sequence. The native antigen may, or may not, contain one or more additional amino acid sequences. A material, molecule, preparation, or the like, which is “isolated” refers to its having been removed from the environment or source in which it naturally occurs.

For example, a polynucleotide sequence which is part of a gene present on a chromosome in a subject or biological source such as an intact, living animal is not isolated, while DNA extracted from a biological sample that has been obtained from such a subject or biological source would be considered isolated. In like fashion, “isolating” may refer to steps taken in the processes or methods for removing such a material from the natural environment in which it occurs.

As used herein, the term “tandem repeat” refers to a region of a polynucleotide sequence (e.g., a sequence of DNA, RNA, recombinantly engineered or synthetic oligonucleotides including linear polymers of non-naturally occurring nucleotides or nucleotide analogs or the like, including nucleotide mimetics) or to a region of a polypeptide or protein comprising a sequence, respectively, of about 6 to 1200 nucleotides or 2 to 400 amino acids, that is repeated in tandem such that the sequence occurs at least two times.

As used herein the term “tandem repeat unit” refers to a single unit of the sequence that is repeated in tandem. Additionally, the term “tandem repeat” also encompasses a region of DNA wherein more than a single 2- to 400-amino acid or 6- to 1200-nucleotide tandem repeat unit is repeated in tandem or with intervening bases or amino acids, provided that at least one of the sequences is repeated at least two times in tandem. In some embodiments, a tandem repeat can have greater than 400 amino acids in a repeat unit or more than 1200 nucleotides in a repeat unit.

Moreover, the term “tandem repeat” also encompasses regions of DNA or a protein wherein the tandem repeat units are not identical. Where two or more sequences are at least 70% homologous to each other or are reasonable variants of each other, these sequences will be considered tandem repeat units for the purpose of comprising and constituting a tandem repeat.

Also, where a sequence is at least 6, 7, 8, 9, 10, 11, 12, 13, 14, 15, 16, 17, 18, 19, 20, 21, 22 or more amino acids of a tandem repeat unit, this sequence may be considered a tandem repeat unit for the purpose of comprising and constituting a tandem repeat.

Also, where a sequence is at least about 18, 21, 24, 27, 30, 33, 36, 39, 42, 45, 48, 51 or any intervening integer of nucleotides, the sequence may be considered a tandem repeat unit for the purpose of comprising and constituting a tandem repeat.

Additionally, the term “tandem repeat” also encompasses tandem repeats where one or more tandem repeat unit of a tandem repeat is the reverse sequence of the other tandem repeat units. Reverse tandem repeat units and non-reverse tandem repeats may be configured in any way and with or without intervening nucleotide bases or amino acids. Configurations of reverse and non-reverse sequences include, but are not limited to, those where a non-reverse sequence is followed by reverse sequence; where a reverse sequence is followed by non-reverse sequence; and where a reverse sequence is followed by a reverse sequence. In the case of double-stranded polynucleotides having tandem repeats two or more such repeats may be present on the same strand or may occur on opposite strands.

In certain embodiments, a tandem repeat may comprise an immunogenic portion of a T. cruzi antigen. An immunogenic portion of a T. cruzi antigen is a portion that is capable of eliciting an immune response (i.e., cellular and/or humoral) in a presently or previously T. cruzi-infected patient (such as a human or a dog) and/or in cultures of lymph node cells or peripheral blood mononuclear cells (PBMC) isolated from presently or previously T. cruzi-infected individuals. Those skilled in the art will be familiar with any of a wide variety of methodologies and criteria for determining whether an immune response has been elicited (See, e.g., Current Protocols in Immunology, John Wiley & Sons Publishers, NY 2000, Chapter 2, Units 2.1-2.3).

The cells in which a response is elicited may comprise a mixture of cell types or may contain isolated component cells (including, but not limited to, T-cells, NK cells, macrophages, monocytes and/or B cells). In particular, immunogenic portions are capable of inducing T-cell proliferation and/or a dominantly Th1-type cytokine response (e.g., IL-2, IFN-γ, and/or TNF-α. production by T-cells and/or NK cells; and/or IL-12 production by monocytes, macrophages and/or B cells). Immunogenic or antigenic portions of the antigens described herein may generally be identified using techniques known to those of ordinary skill in the art, including the representative methods provided herein.

The compositions and methods of various embodiments also encompass variants of the above polypeptides. A polypeptide “variant,” or a polypeptide that is “homologous” to another polypeptide as used herein, is a polypeptide that differs from a native (e.g., naturally occurring) protein in one or more substitutions, deletions, additions and/or insertions, such that the immunogenicity of the polypeptide is not substantially diminished.

For instance, the ability of a variant to react with an antigen-specific antibody, antiserum or T-cell may be enhanced or unchanged, relative to the native protein, or may be diminished by less than 50%, less than 20%, or the like, relative to the native protein. Such variants may generally be identified by modifying one of the herein described polypeptide sequences and evaluating the reactivity of the modified polypeptide with antigen-specific antibodies or antisera as described herein. In one embodiment, variants include those in which one or more portions, such as an N-terminal leader sequence or transmembrane domain, have been removed. Other variants include variants in which a small portion (e.g., 1-30 amino acids or 5-15 amino acids) has been removed from the N— and/or C-terminal of the mature protein.

Polypeptide variants encompassed by various embodiments include those exhibiting at least about 70%, 75%, 80%, 85%, 90%, 91%, 92%, 93%, 94%, 95%, 96%, 97%, 98%, 99% or more identity (determined as described below) to the polypeptides disclosed herein.

In various embodiments, a variant may contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues.

For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In one embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

The polypeptides of various embodiments may be prepared in any suitable manner known in the art. Such polypeptides include naturally occurring polypeptides, recombinantly produced polypeptides, synthetically produced polypeptides, polypeptides produced by a combination of these methods, and the like. Similarly, certain embodiments disclosed herein contemplate polynucleotides comprised of the naturally occurring polynucleotides having sugar- (e.g., ribose or deoxyribose) phosphate backbones in 5′-to-3′ linkage, but the scope of the various embodiments are not so limited and also contemplates polynucleotides comprised of any of a number of natural and/or artificial polynucleotide analogs and/or mimetics, for example; those designed to resist degradation or having other desirably physicochemical properties such as synthetic polynucleotides having a phosphorothioate backbone, and the like.

Polynucleotides may comprise a native sequence (i.e., an endogenous sequence that encodes a protein or a portion thereof) or may comprise a variant, or a biological or antigenic functional equivalent of such a sequence. Polynucleotide variants may contain one or more substitutions, additions, deletions and/or insertions, as further described below, such that the immunogenicity of the encoded polypeptide is not diminished, relative to a native protein. The effect on the immunogenicity of the encoded polypeptide may generally be assessed as described herein. The term “variants” also encompasses homologous genes of xenogenic origin.

When comparing polynucleotide or polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to, in some embodiments, a segment of at least about 20 contiguous positions, sometimes 30 to about 75, sometimes 40 to about 50, sometimes more, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins--Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol.183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. United States 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. United States 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 may be used, for example, with the parameters described herein to determine percent sequence identity for the polynucleotides and polypeptides of some embodiments. Software for performing BLAST analyses is publicly available through the National Center for Biotechnology Information. In one illustrative example, cumulative scores may be calculated using, for nucleotide sequences, the parameters M (reward score for a pair of matching residues; always >0) and N (penalty score for mismatching residues; always <0). For amino acid sequences, a scoring matrix may be used to calculate the cumulative score.

Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a word length (W) of 11, an expectation (E) of 10, and the BLOSUM62 scoring matrix (see Henikoff and Henikoff (1989) Proc. Natl. Acad. Sci. United States 89:10915) alignments, (B) of 50, expectation (E) of 10, M=5, N=−4 and a comparison of both strands.

In one embodiment, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

Therefore, some embodiments encompass polynucleotide and polypeptide sequences having substantial identity to the sequences disclosed herein, for example those comprising at least 50% sequence identity. In some embodiments at least 55%, 60%, 65%, 70%, 75%, 80%, 85%, 90%, 95%, 96%, 97%, 98%, or 99% or higher, sequence identity compared to a polynucleotide or polypeptide sequence of various embodiments using the methods described herein, (e.g., BLAST analysis using standard parameters, as described herein). One skilled in this art will recognize that these values may be appropriately adjusted to determine corresponding identity of proteins encoded by two nucleotide sequences by taking into account codon degeneracy, amino acid similarity, reading frame positioning and the like.

Additional embodiments provide isolated polynucleotides and polypeptides comprising various lengths of contiguous stretches of sequence identical to or complementary to one or more of the sequences disclosed herein. For example, polynucleotides are provided by some embodiments that comprise at least about 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths,” in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

The polynucleotides of some embodiments, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length being limited by the ease of preparation and use in the intended recombinant DNA protocol. For example, illustrative DNA segments with total lengths of about 10,000, about 5,000, about 3,000, about 2,000, about 1,000, about 500, about 200, about 100, about 50 base pairs in length, and the like, (including all intermediate lengths) are contemplated to be useful in many implementations of various embodiments.

In other embodiments, there may be polynucleotides that are capable of hybridizing under moderately stringent conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of some embodiments with other polynucleotides include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2× SSC containing 0.1% SDS.

Moreover, it will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by various embodiments. Further, alleles of the genes comprising the polynucleotide sequences provided herein are within the scope of the embodiments. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

“Polypeptides” as described herein also include combination polypeptides, also referred to as fusion proteins. A “combination polypeptide” or “fusion protein” is a polypeptide comprising at least one of the immunogenic or antigenic portions of a sequence described herein and one or more additional immunogenic sequence, which are joined via a peptide linkage into a single amino acid chain. The sequences may be joined directly (i.e., with no intervening amino acids) or may be joined by way of a linker sequence (e.g., Gly-Cys-Gly) that does not significantly diminish the immunogenic or antigenic properties of the component polypeptides. In various embodiments immunogenic or antigenic sequences may be from T. cruzi.

Fusion proteins may generally be prepared using standard techniques, including chemical conjugation. For example in one embodiment, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in frame. This permits translation into a single fusion protein that retains the biological activity of both component polypeptides.

A peptide linker sequence may be employed to separate the first and the second polypeptide components by a distance sufficient to ensure that each polypeptide folds into its secondary and tertiary structures. Such a peptide linker sequence is incorporated into the fusion protein using standard techniques well known in the art.

Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. In some embodiments, peptide linker sequences contain Gly, Asn and Ser residues. Other near-neutral amino acids, such as Thr and Ala may also be used in the linker sequence.

Amino acid sequences which may be usefully employed as linkers include those disclosed in Maratea et al., Gene 40:39-46,1985; Murphy et al., Proc. Natl. Acad. Sci. United States 83:8258-8262,1986; U.S. Pat. No.4,935,233 and U.S. Pat. No. 4,751,180. The linker sequence may generally be from 1 to about 50 amino acids in length, however in some embodiments may be greater than 50 amino acids. Linker sequences are not required when the first and second polypeptides have non-essential N-terminal amino acid regions that may be used to separate the functional domains and prevent steric interference.

The ligated DNA sequences are operably linked to suitable transcriptional or translational regulatory elements. For example, the regulatory elements responsible for expression of DNA may be located only 5′ to the DNA sequence encoding the first polypeptides. Similarly, stop codons required to end translation and transcription termination signals may be only present 3′ to the DNA sequence encoding the second polypeptide.

In one embodiment, fusion proteins comprise one or more tandem repeat units. In further embodiments, the one or more tandem repeat units are selected from a group consisting of SEQ ID NO 97-192 or have homology to the sequences of SEQ ID NO 97-192. Additionally, various nucleotides may encode such polypeptides, which includes, but is not limited to SEQ ID NO 1-96.

Some embodiments include a fusion protein comprising at least a first and second tandem repeat unit, wherein the first tandem repeat unit comprises an amino acid sequence having at least 8 consecutive amino acids of, and at least 70% homology to, an amino acid sequence selected from the group consisting of SEQ ID NO: 97-192; and wherein the second tandem repeat unit comprises an amino acid sequence having at least 8 consecutive amino acids of, and at least 70% homology to, an amino acid sequence selected from the group consisting of SEQ ID NO: 97-192.

Further embodiments include a fusion protein of wherein the first tandem repeat unit comprises an amino acid sequence having at least 8 consecutive amino acids of, and at least 70% homology to, an amino acid sequence selected from the group consisting of SEQ ID NO: 98-99,101-105 and 110-111; and wherein the second tandem repeat unit comprises an amino acid sequence having at least 8 consecutive amino acids of, and at least 70% homology to, an amino acid sequence selected from the group consisting of SEQ ID NO: 98-99,101-105 and 110-111.

In still further embodiments, a first tandem repeat unit and second tandem repeat unit are identical or the first tandem repeat unit has at least 8 consecutive amino acids of, and at least 70% homology to the second tandem repeat unit.

### Nucleotides According to Various Embodiments

Disclosed herein are exemplary polynucleotides from T. cruzi; however, descriptions of these polynucleotides should not be construed to limit the various embodiments. For example, in some embodiments, polynucleotides may be from various sources of virus or organism as described herein. SEQ ID NO.1-96 disclose exemplary polynucleotides that may encode the polypeptides disclosed in SEQ ID NO. 97-192 respectively. (e.g. in SEQ ID NO.1 encodes in SEQ ID NO. 97; SEQ ID NO.2 encodes in SEQ ID NO. 98 and so forth).

Various embodiments may comprise an isolated polynucleotide that encodes a polypeptide that comprises at least one tandem repeat unit, wherein the tandem repeat unit comprises an amino acid sequence having at least 8 consecutive amino acids of, and at least 70% homology to, an amino acid sequence selected from the group consisting of: SEQ ID NO: 97-192.

Some embodiments may comprise an isolated polynucleotide that encodes a polypeptide that comprises at least two tandem repeat units, wherein each tandem repeat unit comprises an amino acid sequence having at least 8 consecutive amino acids of, and at least 70% homology to, an amino acid sequence selected from the group consisting of: SEQ ID NO: 97-192.

Further embodiments may comprise an isolated polynucleotide that encodes a polypeptide that comprises a fusion protein comprising at least a first and second tandem repeat unit, wherein the first tandem repeat unit comprises an amino acid sequence having at least 8 consecutive amino acids of, and at least 70% homology to, an amino acid sequence selected from the group consisting of SEQ ID NO: 97-192, and wherein the second tandem repeat unit comprises an amino acid sequence having at least 8 consecutive amino acids of, and at least 70% homology to, an amino acid sequence selected from the group consisting of SEQ ID NO: 97-192.

Some embodiments include a nucleotide sequence having at least 24 consecutive nucleotides of, and at least 70% homology to, a sequence selected from the group consisting of: SEQ ID NO: 1-96. Additionally, polynucleotides of various embodiments may be embodied in a recombinant expression vector, a host cell transformed with an expression vector according, and the like.

As discussed herein, there are many nucleotide sequences that may encode a polypeptide as described in SEQ ID NO. 97-192 and according to various embodiments. This may be a result of the degeneracy of the genetic code, among other factors; however, polynucleotides that vary due to differences in codon usage are specifically contemplated in some embodiments.

As describe herein, a “base” or “base-type” refers to a particular type of nucleoside base. Typical bases include adenine, cytosine, guanine, uracil, or thymine bases where the type refers to the sub-population of nucleotides having that base within a population of nucleotide triphosphates bearing different bases. Other rarer bases or analogs may be substituted such as xanthine or hypoxanthine or methylated cytosine. “Nucleoside” includes natural nucleosides, including ribonucleosides and 2′-deoxyribonucleosides, as well as nucleoside analogs having modified bases or sugar backbones.

“Oligonucleotide” or “polynucleotide” refers to a molecule comprised of a plurality of deoxyribonucleotides or nucleoside subunits. The linkage between the nucleoside subunits may be provided by phosphates, phosphonates, phosphoramidates, phosphorothioates, or the like, or by nonphosphate groups as are known in the art, such as peptide-type linkages utilized in peptide nucleic acids (PNAs). The linking groups may be chiral or achiral. The oligonucleotides or polynucleotides may range in length from 2 nucleoside subunits to hundreds or thousands of nucleoside subunits. While oligonucleotides may be 5 to 100 subunits in length, and may also be 5 to 60 subunits in length, the length of polynucleotides may be much greater (e.g., up to 100 kb); however, as described herein, the terms “oligonucleotide” or “polynucleotide” may be used interchangeably unless context dictates otherwise.

The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced (i.e., in the presence of nucleotides and an inducing agent such as DNA polymerase and at a suitable temperature, buffer and pH).

A primer, in some embodiments, may be single stranded for maximum efficiency in amplification, but may alternatively be double stranded. If double stranded, the primer may be first treated to separate its strands before being used to prepare extension products. In some embodiments, the primer is an oligodeoxyribonucleotide. A primer, in various embodiments, must be sufficiently long to prime the synthesis of extension products in the presence of an inducing agent. The exact lengths of the primers depend on many factors, including temperature, source of primer and the use of a given method. In some embodiments a primer may comprise the sequence of SEQ ID NO: 193-200.

A primer may be selected to be “substantially” complementary to a strand of the template or to a specific sequence of the template. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences may be interspersed into the primer, provided that the primer sequence is sufficiently complementary to the sequence of the template to hybridize and thereby form a template primer complex for synthesis of the extension product of the primer. The use of random primers may be used in some embodiments. For example, when the terminal sequence of the target or template polynucleotide is not known, random primer combinations may be used.

The term “nucleotide probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes may be useful in the detection, identification and isolation of particular gene sequences. It is contemplated that a probe used in accordance with various embodiments may be labeled with any “reporter molecule,” so that it is detectable with various detection systems, including, but not limited to fluorescent, enzyme (e.g., ELISA, as well as enzyme-based histochemical assays), radioactive, quantum dots, luminescent systems, and the like. It is not intended that the embodiments be limited to any particular detection system or label.

### Methods of Screening for Antigenic Polypeptide Candidates

In various embodiments, it may be desirable to identify polypeptides that comprise a tandem repeat. In some embodiments, polypeptides comprising a tandem repeat may be identified by screening a proteome for polypeptides that comprise a tandem repeat, screening a genome for genes or nucleotide sequences that encode polypeptides comprising a tandem repeat, and the like. To identify polypeptides that may be antigenic or immunogenic, various criteria may be used in various embodiments to select polypeptides having tandem repeat domains that are likely to be antigenic or immunogenic.

For example, in one embodiment a proteome may be screened and a score may be provided for each polypeptide, or a score may be provided for a given polypeptide sequence based on various criteria, which may include length of tandem repeat unit, homology of tandem repeat units, number of tandem repeat units, hydrophobicity, hydrophilicity, presence of a transmembrane domain, presence of a given signal sequence, and the like.

In another embodiment, a genome may be screened and a score may be provided for a gene or a score may be provided for a given nucleotide sequence based on various criteria, which may include length of tandem repeat unit, homology of tandem repeat units, number of tandem repeat units, hydrophobicity, hydrophilicity, presence of a transmembrane domain, presence of a given signal sequence, and the like. For example, a program such as the Tandem Repeat Finder (G. Benson, “Tandem repeats finder: a program to analyze DNA sequences” Nucleic Acids Research (1999) Vol. 27, No. 2, pp. 573-580) may be used to score and identify nucleotide sequences that may encode a polypeptide comprising a tandem repeat.

In further embodiments, it may be desirable to screen polypeptides based on expression of a given polypeptide during selected developmental cycles of an organism. For example, T. cruzi comprises four developmental stages, namely epimastigote, metacyclic trypomastigote, amastigote, and trypomastigote. Amastigote and trypomastigote stages are primarily found in mammals infected by T. cruzi. Accordingly, it may be desirable to screen for proteins that are expressed in the amastigote and trypomastigote stages because such proteins are more likely to be present in an organism when infected by T. cruzi instead of being only present in non-infectious stages.

In still further embodiments, it may be desirable to identify tandem repeat polypeptides that lack identity to polypeptides found in other disease causing organisms. For example, where immunogenic or antigenic polypeptides are identified in the organism T. cruzi, it may be desirable to screen for polypeptides that lack identity to polypeptides from other organism that cause infectious disease because where polypeptides are used for diagnostic purposes, a false-positive may occur where a given polypeptide has homology among a plurality of disease causing organisms. Comparing polypeptide or nucleotide sequences may be achieved via various methods described herein, and the like. In one embodiment, sequences may be screened for homology to Leishmania.

Additionally, it may be desirable to identify tandem repeat polypeptides that lack identity to polypeptides found in a host organism. For example, where immunogenic or antigenic polypeptides are identified in the organism T. cruzi, it may be desirable to screen for polypeptides that lack identity to polypeptides from H. sapiens because where identified polypeptides are used for diagnostic purposes, a false-positive may occur where human sera is used for diagnostic purposes.

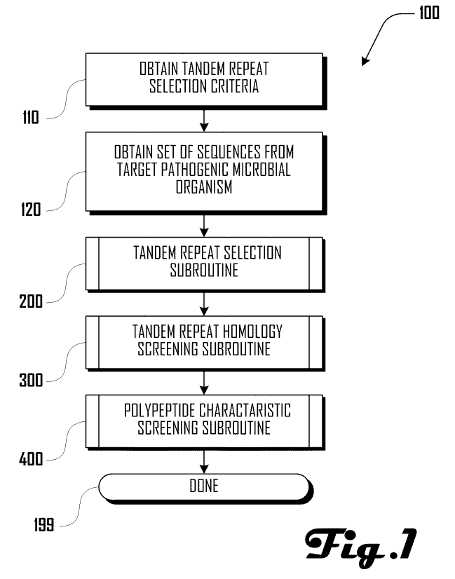

FIG. 1 is a flow diagram illustrating an antigenic polypeptide candidate selection routine 100 in accordance with one embodiment. The antigenic polypeptide candidate selection routine 100 begins in block 110 where tandem repeat selection criteria are obtained and continues to block 120 where a set of sequences is obtained from a target pathogenic microbial organism.

Tandem repeat selection criteria may be any criteria as described herein, such as length of tandem repeat unit, homology of tandem repeat units, number of tandem repeat units, and the like.

In some embodiments, tandem repeat selection criteria or polypeptide characteristic criteria may include hydrophobicity, hydrophilicity, presence of a transmembrane domain, presence of a given signal sequence, and the like. Additionally, sequences may be obtained from one or more organism and may include polypeptide sequences, nucleotide sequences, and the like. In one embodiment, a set of sequences may be obtained from a genome or a proteome.

Returning to the antigenic polypeptide candidate selection routine 100, subroutine block 200 begins a tandem repeat selection subroutine. In subroutine block 300, a sequence homology screening routing begins, and in subroutine block 400 a polypeptide characteristic screening subroutine begins. The antigenic polypeptide candidate selection routine 100 ends in block 199.

In some embodiments, various steps disclosed herein can be absent when selecting one or more tandem repeat polypeptide. For example, in various embodiments, homology screening need not be performed when selecting one or more polypeptide. In another example, polypeptide characteristic screening may be absent. This may be desirable in some embodiments because effective candidate polypeptides may be selected despite having homology to polypeptide or nucleotide sequences of another organism or despite having various characteristics. In further embodiments, the steps depicted in FIG. 1 may be performed in various orders. For example, characteristic screening may occur before tandem repeat homology screening, and the like.

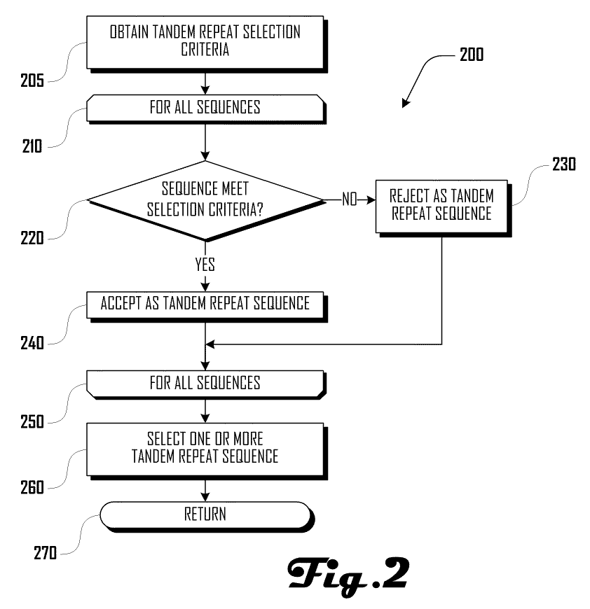

FIG. 2 is a flow diagram illustrating a tandem repeat candidate selection subroutine 200 in accordance with one embodiment. The tandem repeat candidate selection subroutine 200 begins in block 205 where tandem repeat selection criteria are obtained.

Tandem repeat selection criteria may be any criteria as described herein, such as length of tandem repeat unit, homology of tandem repeat units, number of tandem repeat units, and the like. Additionally, sequences may be obtained from one or more organism and may include polypeptide sequences, nucleotide sequences, and the like. In one embodiment, a set of sequences may be, or be obtained from, a genome or a proteome.

Looping block 210 begins a loop for all sequences and in decision block 220 a determination is made whether the sequence meets tandem repeat selection criteria. If the sequence does not meet tandem repeat selection criteria, then the sequence is rejected as a tandem repeat in block 230. However, if the sequence does meet tandem repeat selection criteria, then the sequence is accepted as a tandem repeat in block 240. Looping block 250 ends the loop for all sequences and in block 260 one or more tandem repeat sequence is selected. The subroutine then returns 270 to its calling routine.

For example, in one embodiment, sequences from a genome or proteome may be screened to determine if the sequences meet tandem repeat selection criteria, and then one or more of the sequences identified as a tandem repeat may then be selected for further screening. In a further embodiment, a genome or proteome may be searched generally for sequences that may be tandem repeat sequences.

### Homology Screening Methods

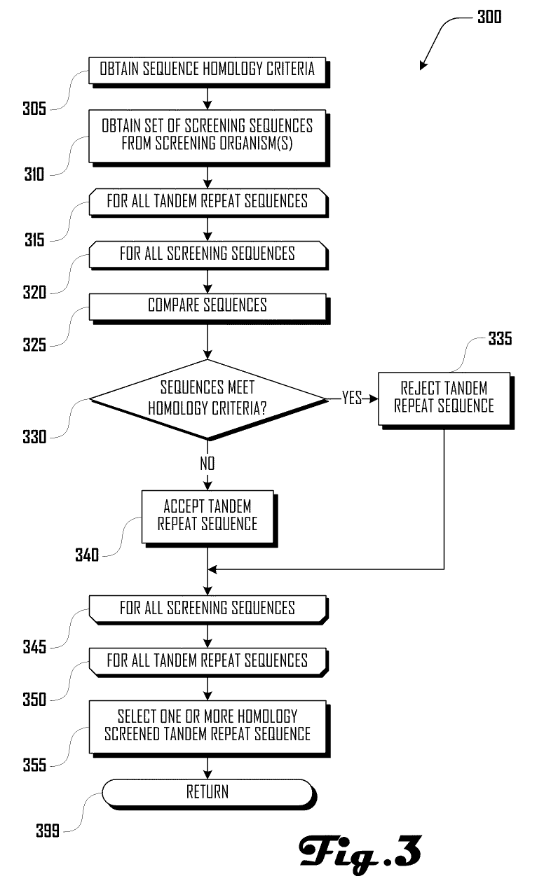

FIG. 3 is a flow diagram illustrating a tandem repeat candidate homology screening subroutine 300 in accordance with one embodiment.

Sequence homology criteria are obtained and in block 305 and in block 310, a set of screening sequences from one or more organism is obtained. Sequence homology criteria may include defining a threshold of maximum allowed homology, which may be expressed in percentage homology such as 10%, 20%, 30%, 40%, 50%, 55%, 60%, 65%, 70%, 80%, 85%, 90%, 95%, 98%, 99% and the like.

Additionally, methods of determining sequence homology and comparing sequences are well known in the art and such various methods are within the scope of various embodiments. For example, tools or programs that may be used to compare sequences include BLAST search of various types such as blastn, blastp, PSI-BLAST, blastx, tblastx, tblastn, megablast, BLAT, BLASTZ, Progeniq, FPGA-BLAST, Tera-BLAST, and the like. (Altschul S F, Gish W, Miller W, Myers E W, Lipman D J (1990); see also, www.ncbi.nim.hih.gov/BLAST).

Looping block 315 begins a loop for all tandem repeat sequences and looping block 320 begins a loop for all screening sequences. In block 325 the tandem repeat sequence is compared to the screening sequence and in decision block 330 a determination is made whether the sequences meet homology criteria.

If the sequence meets homology criteria, then the tandem repeat sequence is rejected in block 335; however, if the sequence does not meet homology criteria, then the tandem repeat sequence is accepted in block 340. The tandem repeat homology screening subroutine 300 continues to looping block 345 which terminates the loop for all screening sequences and continues to looping block 350, which terminates the loop for all tandem repeat sequences. In block 355 one or more homology screened tandem repeat sequence is selected and the tandem repeat homology screening subroutine 300 returns 399 to its calling routine.

For example, a set of selected tandem repeat sequences, either nucleotide or polypeptide sequences, may be selected and screened for homology to sequences from another organism. In some embodiments, portions of an entire genome or proteome from one or more organism, other than the target organism, may be screened against. In various embodiments, tandem repeat sequences may be screened against sequences from a host organism or against sequences of an infectious organism. In further embodiments, an entire sequence or one or more portion of a sequence may be used for screening.

### Polypeptide Characteristic Screening Methods

In some embodiments, tandem repeat selection criteria or polypeptide characteristic criteria may include hydrophobicity, hydrophilicity, presence of a transmembrane domain, presence of a given signal sequence, molecular mass, isoelectric point, life cycle presence, and the like. For example, it may be desirable to screen for polypeptides having characteristics which make them more likely to be antigenic.

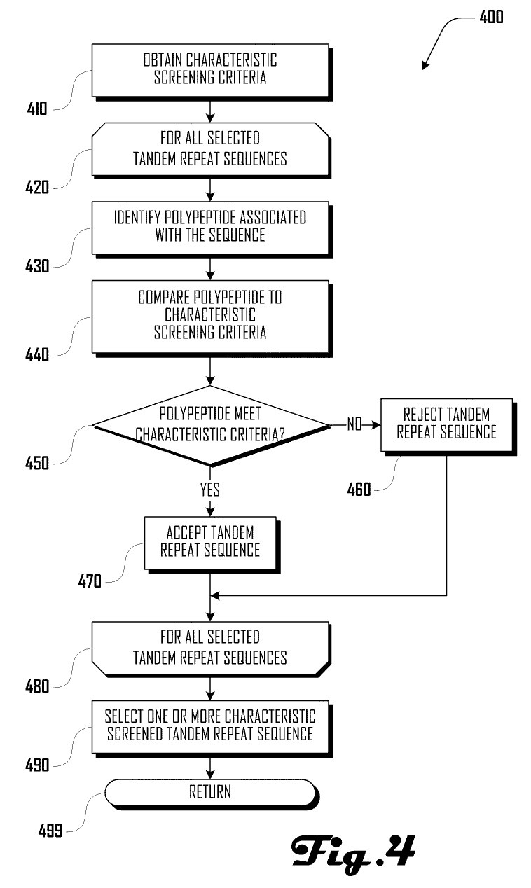

FIG. 4 is a flow diagram illustrating an antigenic tandem repeat candidate characteristic screening subroutine 400 in accordance with one embodiment. The antigenic tandem repeat candidate characteristic screening subroutine 400 begins in block 410 where characteristic screening criteria are obtained. Looping block 420 beings a loop for all selected tandem repeat sequences. In block 430, a polypeptide associated with a sequence is identified.

For example, identification of a polypeptide may include comparing a sequence to a proteome to determine if the sequence is present in any known polypeptides, hypothetical polypeptides, and the like. Additionally, polypeptides may be selected which are homologous to the selected sequence.

Returning to the antigenic tandem repeat candidate characteristic screening subroutine 400, an identified polypeptide is compared to polypeptide characteristic screening criteria and in decision block 450, a determination is made whether the polypeptide meets the characteristic criteria.

If the polypeptide does not meet the characteristic screening criteria, then the antigenic tandem repeat candidate characteristic screening subroutine 400 continues to block 460 where the tandem repeat sequence associated with the polypeptide is rejected, and the antigenic tandem repeat candidate characteristic screening subroutine 400 continues to looping block 480 where the loop is ended for all selected tandem repeat sequences.

However, if the identified polypeptide does meet the characteristic screening criteria, the antigenic tandem repeat candidate characteristic screening subroutine 400 continues to looping block 480 where the loop is ended for all selected tandem repeat sequences.

In block 490, one or more characteristic screened tandem repeat sequences is selected and the antigenic tandem repeat candidate characteristic screening subroutine 400 returns to its calling routine in block 499.

### Methods of Screening for Antigenic Polypeptides

In addition to screening for antigenic polypeptide candidates, various embodiments include screening for antigenic or immunogenic polypeptides. Such screening may be performed on selected antigenic polypeptide candidates as discussed herein, or may be performed on genomes, proteomes, or biological samples.

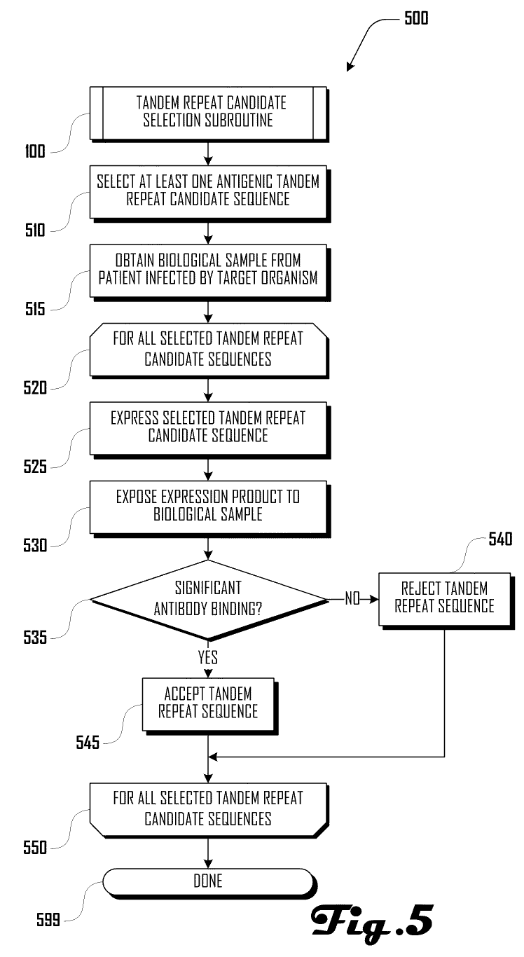

For example, FIG. 5 depicts a flow diagram illustrating an antigenic tandem repeat polypeptide screening routine 500 in accordance with one embodiment, which is performed on antigenic polypeptides via methods of identifying antigenic polypeptide candidates as described in FIGS. 1-4.

Accordingly, the antigenic tandem repeat polypeptide screening routine 500 begins in subroutine block 100, where tandem repeat candidates are selected. In block 510, at least one antigenic tandem repeat candidate sequence is selected from the set obtained from block 100. In block 515 a biological sample is obtained from a patient infected by a target infectious organism. Such a biological sample may include blood, sera, urine, cerebrospinal fluid, and the like. In various embodiments, a biological sample may be chosen based on presence of antibodies to the target infectious organism.

Looping block 520 begins a loop for all selected tandem repeat candidate sequences and in block 525 the antigenic tandem repeat candidate sequence is expressed and in block 530 the expression product is exposed to the biological sample.

In various embodiments, exposure of the expression product to the biological sample may include various conditions sufficient to allow antibodies present in the biological sample to selectively bind with the expression product. In decision block 535 a determination is made whether significant binding has occurred which may be based on a pre-defined threshold of detected binding activity. Such binding activity may be detected via methods known in the art or methods as described herein.

If significant binding has not occurred, the antigenic tandem repeat candidate sequence is rejected in block 540 and block 550 ends the loop for all selected antigenic tandem repeat candidate sequences. However, if significant binding has occurred, then the antigenic tandem repeat sequence candidate is accepted in block 545 and block 550 ends the loop for all selected antigenic tandem repeat candidate sequences. The antigenic tandem repeat polypeptide screening routine 500 ends in block 599.

### Compounds, Systems and Methods for Detecting Chagas Disease and the Like

As described above, some embodiments disclose methods of screening and selecting tandem repeat proteins that may have efficacy in detecting an infectious disease such as Chagas disease, and the like. Polypeptides screened and selected by this and other methods of various embodiments may be used for various applications, including but not limited to systems and methods for detecting, treating, preventing, monitoring and immunizing against an infection in organisms, blood supplies, and the like. In various exemplary embodiments, such systems and methods are directed to Chagas disease.

Methods of detecting T. cruzi according to some embodiments includes contacting a biological sample from a subject suspected of having a T. cruzi infection with a polypeptide, wherein the polypeptide comprises at least one tandem repeat unit, wherein the tandem repeat unit comprises an amino acid sequence having at least 8 consecutive amino acids of, and at least 70% homology to, an amino acid sequence selected from the group consisting of: SEQ ID NO: 97-192, under conditions and for a time sufficient for binding of an antibody in the sample to take place; and detecting in the biological sample the presence of at least one T. cruzi specific antibody specifically bound to the polypeptide, thereby detecting T. cruzi infection in the subject.

Further embodiments include methods wherein the polypeptide comprises at least two tandem repeat units, wherein each tandem repeat unit comprises an amino acid sequence having at least 8 consecutive amino acids of, and at least 70% homology to, an amino acid sequence selected from the group consisting of: SEQ ID NO: 97-192. Additionally, the first and second tandem repeat unit may be identical, and the first tandem repeat unit may comprise at least 8 consecutive amino acids of, and at least 70% homology to the second tandem repeat unit.

Various embodiments include a diagnostic kit for detecting T. cruzi infection in a biological sample, comprising a plurality of polypeptides, wherein each of the plurality of polypeptides comprises at least one tandem repeat unit, wherein the tandem repeat unit comprises an amino acid sequence having at least 8 consecutive amino acids of, and at least 70% homology to, an amino acid sequence selected from the group consisting of: SEQ ID NO: 97-192; and a detection reagent. Additionally, polypeptides may be covalently or non-covalently bound to a solid support and the solid support may comprise materials such as nitrocellulose, latex or a plastic material.

In one exemplary embodiment, methods are disclosed for detecting and monitoring T. cruzi infection in individuals, blood supplies, and other organisms. In general, T. cruzi infection may be detected in any biological sample that contains antibodies. For example, a biological sample may be blood, serum, plasma, saliva, cerebrospinal fluid, urine, and the like. In various embodiments, the sample is a blood or serum sample obtained from a patient or a blood supply. Briefly, T. cruzi infection may be detected using one or more tandem repeat polypeptides, fusion proteins or other polypeptides as discussed herein, or variants thereof. The one or more tandem repeat polypeptides, fusion proteins or other polypeptides may then be used to determine the presence or absence of antibodies that are capable of specifically binding to a polypeptide in the sample.

Polypeptides within the scope of various embodiments include, but are not limited to, polypeptides comprising immunogenic or antigenic portions of T. cruzi antigens comprising the sequences recited in SEQ ID NO: 97-192. Additionally, SEQ ID NO: 1-96 comprise nucleotides that may encode proteins or polypeptides that are in accordance with various embodiments. As used herein, the term “tandem repeat” in some embodiments, refers to a region of DNA or a protein comprising a sequence of 3 to 400 nucleotides, or more, or 1 to 200 amino acids, or more repeated in tandem at least two times. As used herein, the term “tandem repeat unit” refers to a single unit of the sequence that is repeated in tandem.

As used herein, references to “binding” interactions between two molecules, such as between an antibody and its cognate antigen, may include binding that may, according to non-limiting theory, be the result of one or more of electrostatic interactions, hydrophobic interactions, steric interactions, van der Waals forces, hydrogen bonding or the like, or other types of interactions influencing such binding events, such as binding of an antibody to a polypeptide, binding of a detection reagent to an antibody/peptide complex, or any other binding interaction of molecules, including in some embodiments specific binding interactions wherein the “specific” binding affinity constant, Ka, may typically be less than about 10-9 M, less than about 10-8 M, less than about 10-7 M, less than about 10-6 M, less than about 10-5 M or less than 10-4 M.

There are a variety of assay formats known to those of ordinary skill in the art for using a polypeptide to detect antibodies in a sample. See, e.g., Current Protocols in Immunology (Coligan et al., eds., John Wiley & Sons, publishers), and Harlow and Lane, Antibodies. A Laboratory Manual, Cold Spring Harbor Laboratory, 1988, which are incorporated herein by reference. In one embodiment, the assay involves the use of a polypeptide (e.g., a polypeptide antigen comprising one or more tandem repeat units as described herein) immobilized on a solid support to bind to and remove the antibody from the sample. The bound antibody may then be detected using a detection reagent that specifically binds to the antibody/polypeptide complex, and that comprises a readily detectable moiety such as a detectable reporter group. Suitable detection reagents include antibodies that bind to the antibody/polypeptide complex and free polypeptide labeled with a reporter group (e.g., in a semi-competitive assay).

Alternatively, a competitive assay may be utilized, in which an antibody that binds to the polypeptide is labeled with a reporter group and allowed to bind to the immobilized polypeptide after incubation of the polypeptide with the sample. The extent to which components of the sample inhibit the binding of the labeled antibody to the polypeptide is indicative of the reactivity of the sample with the immobilized polypeptide.

The solid support may be any material known to those of ordinary skill in the art to which the polypeptide may be attached. For example, the support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681.

The polypeptide may be bound to the solid support using a variety of techniques known to those in the art, which are amply described in the patent and scientific literature. In the context of some embodiments, the term “bound” refers to both non-covalent association, such as adsorption, and covalent attachment (which may be a direct linkage between the antigen and functional groups on the support or may be a linkage by way of a cross-linking agent).

Binding by adsorption to a well in a microtiter plate or to a membrane is contemplated in one embodiment. In such cases, adsorption may be achieved by contacting the polypeptide, in a suitable buffer, with the solid support for a suitable amount of time. The contact time can vary with temperature, but is typically between about 1 hour and 1 day. In some embodiments, contact time can be less than an hour, and may be a number of minutes or second. In general, contacting a well of a plastic microtiter plate (such as polystyrene or polyvinylchloride) with an amount of polypeptide ranging from about 10 ng to about 1 μg, and alternatively about 100 ng, is sufficient to bind an adequate amount of antigen. Nitrocellulose will bind approximately 100 μg of protein per cm³.

Covalent attachment of polypeptides to a solid support may generally be achieved by first reacting the support with a bifunctional reagent that will react with both the support and a functional group, such as a hydroxyl or amino group, on the polypeptide. For example, the polypeptide may be bound to a support having an appropriate polymer coating using benzoquinone or by condensation of an aldehyde group on the support with an amine and an active hydrogen on the polypeptide (see, e.g., Pierce Immunotechnology Catalog and Handbook (1991) at A12-A13).

In certain embodiments, the assay is an Enzyme Linked ImmunoSorbent Assay (“ELISA”). This assay may be performed by first contacting a polypeptide antigen that has been immobilized on a solid support, commonly the well of a microtiter plate, with the sample, such that antibodies to the polypeptide within the sample are allowed to bind to the immobilized polypeptide. Unbound sample is then removed from the immobilized polypeptide and a detection reagent capable of binding to the immobilized antibody-polypeptide complex is added. The amount of detection reagent that remains bound to the solid support is then determined using a method appropriate for the specific detection reagent.

Once the polypeptide is immobilized on the support, the remaining protein binding sites on the support are typically blocked. Any suitable blocking agent known to those of ordinary skill in the art, such as bovine serum albumin (BSA) or Tween 20™ (Sigma Chemical Co., St. Louis, Mo.) may be employed. The immobilized polypeptide is then incubated with the sample, and antibodies (if present in the sample) are allowed to bind to the antigen. The sample may be diluted with a suitable diluent, such as phosphate-buffered saline (PBS), prior to incubation. In general, an appropriate contact time (i.e., incubation time) is that period of time that is sufficient to permit detection of the presence of antibody within a T. cruzi-infected sample. In one embodiment, the contact time is sufficient to achieve a level of binding that is at least 95% of that achieved at equilibrium between bound and unbound antibodies. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 20.™ Detection reagent may then be added to the solid support. An appropriate detection reagent is any compound that binds to the immobilized antibody-polypeptide complex and that may be detected by any of a variety of means known to those in the art. For example, in one embodiment, the detection reagent contains a binding agent (such as, for example, Protein A, Protein G, immunoglobulin, lectin or free antigen) conjugated to a reporter group. In some embodiments, reporter groups include enzymes (such as horseradish peroxidase), substrates, cofactors, inhibitors, dyes, radionuclides, luminescent groups, fluorescent groups and biotin. The conjugation of binding agent to reporter group may be achieved using standard methods known to those of ordinary skill in the art. Common binding agents may also be purchased conjugated to a variety of reporter groups from many sources (e.g., Zymed Laboratories, San Francisco, Calif. and Pierce, Rockford, Ill.).

The detection reagent is then incubated with the immobilized antibody-polypeptide complex for an amount of time sufficient to detect the bound antibody. An appropriate amount of time may generally be determined from the manufacturer's instructions or by assaying the level of binding that occurs over a period of time. Unbound detection reagent is then removed and the bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of anti-T. cruzi antibodies in the sample, the signal detected from the reporter group that remains specifically bound to the solid support is generally compared to a signal that corresponds to an appropriate control according to art-accepted methodologies, for example, a predetermined cut-off value. In one embodiment, the cut-off value may be the average mean signal obtained when the immobilized polypeptide is incubated with samples from an uninfected patient. In general, a sample generating a signal that is three standard deviations above the predetermined cut-off value is considered positive (i.e., reactive with the polypeptide). In an alternate embodiment, the cut-off value is determined using a Receiver Operator Curve, according to the method of Sackett et al., Clinical Epidemiology: A Basic Science for Clinical Medicine, p. 106-7 (Little Brown and Co., 1985).

Briefly, in such an embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate.

In a related embodiment, the assay is performed in a flow-through or strip test format, wherein the antigen (e.g., one or more polypeptides, each comprising at least one tandem repeat unit) is immobilized on a solid support, for instance, a membrane such as nitrocellulose. In a flow-through test, the fluid sample is contacted with the solid support under conditions and for a time sufficient to permit antibodies, if present within the sample, to bind specifically to the immobilized polypeptide as the sample passes through the membrane. A detection reagent (e.g., protein A-colloidal gold) that may be present in the solid support, or that may alternatively be applied, then binds to the antibody-polypeptide complex as the solution containing the detection reagent flows through the membrane.

Determination of bound detection reagent may then be performed as described above. In certain related embodiments of the strip test format, one end of a solid support membrane to which the polypeptide antigen is bound is immersed in a solution containing the sample. The sample migrates along the membrane through a region containing detection reagent and to the area of immobilized polypeptide antigen. Concentration of detection reagent at the area of the immobilized polypeptide antigen indicates the presence of T. cruzi antibodies in the sample.

Typically, the concentration of detection reagent at that site generates a pattern, such as a line or a series of two or more lines, which may be read visually. The absence of such a pattern indicates a negative result. In general, the amount of polypeptide immobilized on the membrane is selected to generate a visually discernible pattern when the biological sample contains a level of antibodies that would be sufficient to generate a positive signal in an ELISA, as discussed above. In one embodiment, the amount of polypeptide immobilized on the membrane ranges from about 25 ng to about 1 μg, and alternatively from about 50 ng to about 500 ng. Such tests may typically be performed with a very small amount (e.g., one drop) of patient serum or blood.

Clearly, numerous other assay protocols exist that are suitable for use with the polypeptides of various embodiments, and these will be known to those familiar with the art for detecting the presence of an antibody that is capable of specifically binding to a particular polypeptide antigen. The above descriptions are intended to be exemplary only.

Systems and Methods of Treating, Preventing & Immunizing Against Diseases such as Chagas Disease

As described above, various embodiments disclose methods of screening and selecting tandem repeat polypeptides that may have efficacy in detecting an infection in a biological sample or organism. Polypeptides screened and selected by this and other methods as described herein may be used for various applications, including but not limited to systems and methods for detecting, treating, preventing, monitoring and immunizing against Chagas disease, and other infectious diseases, in organisms or blood supplies.

Accordingly, in certain aspects of various embodiments, described in detail below, the polypeptides, antigenic epitopes, tandem repeat units, immunogenic sequences, fusion proteins and/or soluble antigens may be incorporated into pharmaceutical compositions or vaccines. For clarity, the term “polypeptide” will be used when describing specific embodiments of the inventive therapeutic compositions and diagnostic methods; however, it will be clear to one of skill in the art that the antigenic epitopes, polypeptides, tandem repeat units and fusion proteins of some embodiments may also be employed in such compositions and methods.

Pharmaceutical compositions may comprise one or more polypeptides, each of which may contain one or more of the above sequences (or variants thereof), and a physiologically acceptable carrier. Vaccines, also referred to as immunogenic compositions, may comprise one or more of the above polypeptides, such as a polypeptide of SEQ ID NO: 97-192 or a polypeptide encoded by, expressed from, or originating from a nucleotide of SEQ ID NO: 1-96 and an immunostimulant, such as an adjuvant (e.g., LbeIF4A, interleukin-12 or other cytokines) or a liposome (into which the polypeptide is incorporated).

Many adjuvants contain a substance designed to protect the antigen from rapid catabolism, such as aluminum hydroxide or mineral oil, and a stimulator of immune responses, such as lipid A, Bordetella pertussis or Mycobacterium tuberculosis derived proteins. Certain adjuvants are commercially available, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2,-7,-12, and other like growth factors, may also be used as adjuvants.

Within certain embodiments, it may be desirable for the adjuvant composition to be such that it induces an immune response predominantly of the Th1 type. By virtue of the ability to induce an exclusive Th1 immune response, the use of LbeIF4A, and variants thereof, as an adjuvant in the vaccines various embodiments may be desirable. Certain other adjuvants for eliciting a predominantly Th1-type response include, for example, Imiquimod, Res-Imiquimod, a combination of monophosphoryl lipid A, alternatively 3-de-O-acylated monophosphoryl lipid A, together with an aluminum salt. MPL® adjuvants are available from Corixa Corporation/Glaxo Smith Kline (see, for example, U.S. Pat. Nos. 4,436,727; 4,877,611; 4,866,034 and 4,912,094).

CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352,1996. Other adjuvants, according to other embodiments, may comprise a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other formulations include more than one saponin in the adjuvant combinations of some embodiments, for example combinations of at least two of the following group comprising QS21, QS7, Quil A, β-escin, or digitonin.

Alternatively the saponin formulations may be combined with vaccine vehicles composed of chitosan or other polycationic polymers, polylactide and polylactide-co-glycolide particles, poly-N-acetyl glucosamine-based polymer matrix, particles composed of polysaccharides or chemically modified polysaccharides, liposomes and lipid-based particles, particles composed of glycerol monoesters, etc. The saponins may also be formulated in the presence of cholesterol to form particulate structures such as liposomes or ISCOMs.

Furthermore, the saponins may be formulated together with a polyoxyethylene ether or ester, in either a non-particulate solution or suspension, or in a particulate structure such as a paucilamelar liposome or ISCOM. The saponins may also be formulated with excipients such as Carbopol® to increase viscosity, or may be formulated in a dry powder form with a powder excipient such as lactose.

In one embodiment, the adjuvant system may include the combination of a monophosphoryl lipid A and a saponin derivative, such as the combination of QS21 and 3D-MPL® adjuvant, as described in WO 94/00153, or a less reactogenic composition where the QS21 is quenched with cholesterol, as described in WO 96/33739. Other formulations comprise an oil-in-water emulsion and tocopherol. In another exemplary adjuvant formulation, employing QS21, 3D-MPL® adjuvant and tocopherol in an oil-in-water emulsion may be used, such as described in WO 95/17210.

A further adjuvant system, in accordance with another embodiment, involves the combination of a CpG-containing oligonucleotide and a saponin derivative particularly the combination of CpG and QS21 is disclosed in WO 00/09159. For example, the formulation may additionally comprise an oil-in-water emulsion and tocopherol.

Additional illustrative adjuvants for use in the compositions of various embodiments may include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), EnhanZyn™ (Corixa, Hamilton, Mont.), RC-529 (Corixa, Hamilton, Mont.) and other aminoalkyl glucosaminide 4-phosphates (AGPs), such as those described in U.S. Pat. No. 6,113,918 and U.S. patent application Ser. No. 09/074,720, the disclosures of which are incorporated herein by reference in their entireties, and polyoxyethylene ether adjuvants such as those described in WO 99/52549A1.

Other adjuvants include adjuvant molecules of the general formula (I): HO(CH₂CH₂O)ₙ-A--R, wherein, n is 1-50, A is a bond or —C(O)—, R is C₁₋₅₀ alkyl or Phenyl C₁₋₅₀ alkyl. One embodiment consists of a vaccine formulation comprising a polyoxyethylene ether of general formula (I), wherein n is between 1 and 50; in some embodiment 4-24; the R component is C₁₋₅₀, such as C₄-C₂₀ alkyl and in some embodiments C₁₂ alkyl, and A is a bond. The concentration of the polyoxyethylene ethers may be in the range 0.1-20%, and in some embodiments from 0.1-10%, and in other embodiments in the range 0.1-1%.

Polyoxyethylene ethers may be polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, polyoxyethylene-23-lauryl ether, and the like. Polyoxyethylene ethers such as polyoxyethylene lauryl ether are described in the Merck index (12th edition: entry 7717). These adjuvant molecules are described in WO 99/52549. The polyoxyethylene ether according to the general formula (I) above may, if desired, be combined with another adjuvant. For example, one adjuvant combination is with CpG as described in the UK patent application GB 9820956.2.

Vaccines may additionally contain a delivery vehicle, such as a biodegradable microsphere (disclosed, for example, in U.S. Pat. Nos. 4,897,268 and 5,075,109). Pharmaceutical compositions and vaccines within the scope of various embodiments may also contain other antigens or other T. cruzi antigens, either incorporated into a combination polypeptide or present within one or more separate polypeptides.

Alternatively, a pharmaceutical or immunogenic composition may contain an immunostimulant, such as an adjuvant (e.g., LbeIF4A, interleukin-12 or other cytokines, or DNA coding for such enhancers), and a polynucleotide (e.g., DNA) encoding one or more of the polypeptides or fusion proteins described above, such that the polypeptide is generated in situ. In such compositions, the DNA may be present within any of a variety of delivery systems known to those of ordinary skill in the art, including nucleic acid expression systems, and bacterial and viral expression systems. Appropriate nucleic acid expression systems contain the necessary DNA sequences for expression in the patient (such as a suitable promoter and terminating signal).

Bacterial delivery systems involve the administration of a bacterium (such as Bacillus-Calmette-Guerrin) that expresses an immunogenic portion of the polypeptide on its cell surface. In one embodiment, the DNA may be introduced using a viral expression system (e.g., vaccinia or other pox virus, retrovirus, or adenovirus), which may involve the use of a non-pathogenic (defective) replication competent virus. Techniques for incorporating DNA into such expression systems are well known to those of ordinary skill in the art. The DNA may also be “naked,” in some embodiments, as described, for example, in Ulmer et al., Science 259:1745-1749 (1993) and reviewed by Cohen, Science 259:1691-1692 (1993). The uptake of naked DNA may be increased by coating the DNA onto biodegradable beads, which are efficiently transported into the cells.

While any suitable carrier known to those of ordinary skill in the art may be employed in the pharmaceutical compositions of various embodiments, the type of carrier will vary depending on the mode of administration. For parenteral administration, such as subcutaneous injection, the carrier may comprise, for example, water, saline, alcohol, a fat, a wax, a buffer, and the like. For oral administration, any of the above carriers or a solid carrier, such as mannitol, lactose, starch, magnesium stearate, sodium saccharine, talcum, cellulose, glucose, sucrose, magnesium carbonate, and the like, may be employed. Biodegradable microspheres (e.g., polylactic galactide) may also be employed as carriers for the pharmaceutical compositions of various embodiments. Suitable biodegradable microspheres are disclosed, for example, in U.S. Pat. Nos. 4,897,268 and 5,075,109.

In one embodiment, compositions include multiple polypeptides selected so as to provide enhanced protection against a variety of organism species that cause a given disease. Such polypeptides may be selected based on the species of origin of the native antigen or based on a high degree of conservation of amino acid sequences among different species of the genus.

A combination of individual polypeptides may be particularly effective as a prophylactic and/or therapeutic vaccine in some embodiments because (1) stimulation of proliferation and/or cytokine production by a combination of individual polypeptides may be additive, (2) stimulation of proliferation and/or cytokine production by a combination of individual polypeptides may be synergistic, (3) a combination of individual polypeptides may stimulate cytokine profiles in such a way as to be complementary to each other and/or (4) individual polypeptides may be complementary to one another when certain of them are expressed more abundantly on the individual species or strain of organism responsible for infection. For example, in one embodiment, various strains of T. cruzi. Alternatively, or in addition, the combination may include one or more polypeptides comprising immunogenic portions of other antigens disclosed herein, and/or soluble antigens.

In another embodiment, compositions include single polypeptides selected so as to provide enhanced protection against a variety of species. A single individual polypeptide may be particularly effective as a prophylactic and/or therapeutic vaccine for those reasons stated above for combinations of individual polypeptides.

In another embodiment, compositions include individual polypeptides and combinations of the above described polypeptides employed with a variety of adjuvants, such as IL-12 (protein or DNA) to confer a protective response against a variety of species within a given genus.

In yet another embodiment, compositions include DNA constructs of the various species within a genus employed alone or in combination with a variety of adjuvants, to confer a protective response against a variety of species within a given genus.

The above pharmaceutical compositions and vaccines may be used, for example, to induce protective immunity against a given disease in a patient, such as a human or a dog, to prevent the disease. In various embodiments, the disease may be Chagas disease. Appropriate doses and methods of administration for these purposes are described in detail below.

The pharmaceutical and immunogenic compositions described herein may also be used to stimulate an immune response, which may be cellular and/or humoral, in a patient. For example, in infected patients, the immune responses that may be generated include Th1 immune response (i.e., a response characterized by the production of the cytokines interleukin-1, interleukin-2, interleukin-12 and/or interferon-γ, as well as tumor necrosis factor-α).

For uninfected patients, the immune response may be the production of interleukin-12 and/or interleukin-2, or the stimulation of gamma delta T-cells. In either category of patient, the response stimulated may include IL-12 production. Such responses may also be elicited in biological samples of PBMC or components thereof derived from infected or uninfected individuals. As noted above, assays for any of the above cytokines may generally be performed using methods known to those of ordinary skill in the art, such as an enzyme-linked immunosorbent assay (ELISA) as described herein.

Suitable pharmaceutical compositions and vaccines for use in this aspect of various embodiments are those that contain at least one polypeptide comprising an immunogenic portion of an antigen as disclosed herein (or a variant thereof). In some embodiments, the polypeptides employed in the pharmaceutical compositions and vaccines are complementary, as described above. Soluble antigens, with or without additional polypeptides, may also be employed. In one embodiment, antigens may be from T. cruzi, which may include one or more of SEQ ID NO: 97-192 or a variation thereof.

The pharmaceutical compositions and vaccines described herein may also be used to treat a patient afflicted with a disease responsive to IL-12 stimulation. The patient may be any warm-blooded animal, such as a human, dog, rodent, or the like. Such diseases include infections (which may be, for example, bacterial, viral or protozoan) or diseases such as cancer. In one embodiment, the disease is Chagas disease, and the patient may display clinical symptoms or may be asymptomatic.

In general, the responsiveness of a particular disease to IL-12 stimulation may be determined by evaluating the effect of treatment with a pharmaceutical composition or vaccine of various embodiments on clinical correlates of immunity. For example, if treatment results in a heightened Th1 response or the conversion of a Th2 to a Th1 profile, with accompanying clinical improvement in the treated patient, the disease is responsive to IL-12 stimulation. Polypeptide administration may be as described below, or may extend for a longer period of time, depending on the indication. In one embodiment, the polypeptides employed in the pharmaceutical compositions and vaccines are complementary, as described above. In a further embodiment, a combination contains polypeptides that comprise immunogenic portions of LmSTI1, Ldp23, Lbhsp83, Lt-1 and LbeIF4A, Lmsp1a, Lmsp9a, and TSA. Soluble antigens, with or without additional polypeptides, may also be employed.

Routes and frequency of administration, as well as dosage, for the above aspects of some embodiments will vary from individual to individual and may parallel those currently being used in immunization against other infections, including protozoan, viral and bacterial infections. In general, the pharmaceutical compositions and vaccines may be administered by injection (e.g., intracutaneous, intramuscular, intravenous or subcutaneous), intranasally (e.g., by aspiration) or orally. In some embodiments, between 1 and 12 doses may be administered over a 1 year period.

For therapeutic vaccination, in accordance with other embodiments, (i.e., treatment of an infected individual), 12 doses are administered at one month intervals. For prophylactic use, for example, 3 doses are administered at 3 month intervals. In either case, booster vaccinations may be given periodically thereafter. Alternate protocols may be appropriate for individual patients.

In accordance in one embodiment, a suitable dose is an amount of polypeptide or nucleotide that, when administered as described above, is capable of raising an immune response in an immunized patient sufficient to protect the patient from a disease for at least 1-2 years. In various embodiments, the amount of polypeptide present in a dose (or produced in situ by the DNA in a dose) ranges from about 100 ng to about 1 mg per kg of host, typically from about 10 μg to about 100 μg. Suitable dose sizes will vary with the size of the patient, but will typically range from about 0.1 mL to about 5 mL. One of ordinary skill in the art will immediately appreciate that various dosage sizes may be possible, which are within the scope and spirit of various embodiments.

In another aspect, some embodiments provide methods of using one or more of the polypeptides described above to diagnose an infection, such as a T. cruzi infection, in a patient using a skin test. As used herein, a “skin test” is any assay performed directly on a patient in which a delayed-type hypersensitivity (“DTH”) reaction (such as induration and accompanying redness) is measured following intradermal injection of one or more polypeptides as described above. Such injection may be achieved using any suitable device sufficient to contact the polypeptide or polypeptides with dermal cells of the patient, such as a tuberculin syringe or 1 mL syringe. For example, in some embodiments, the reaction is measured at least 48 hours after injection, or alternatively 72 hours after injection.

The DTH reaction is a cell-mediated immune response, which is greater in patients that have been exposed previously to a test antigen (i.e., an immunogenic portion of a polypeptide employed, or a variant thereof). The response may be measured visually using a ruler. In some embodiments, induration that is greater than about 0.5 cm in diameter, and in other embodiments greater than about 1.0 cm in diameter, is a positive response, indicative of infection, which may or may not be manifested as an active disease.

The polypeptides of some embodiments may be formulated, for use in a skin test, as pharmaceutical compositions containing at least one polypeptide and a physiologically acceptable carrier, as described above. Such compositions, in some embodiments, contain one or more of the above polypeptides in an amount ranging from about 1 μg to 100 μg, and in other embodiments from about 10 μg to 50 μg in a volume of 0.1 mL. For example, the carrier employed in such pharmaceutical compositions may be a saline solution with appropriate preservatives, such as phenol and/or Tween₈₀T.

The polypeptides of SEQ ID NOS: 97-192 may also be employed in combination with one or more known T. cruzi antigens in the diagnosis of Chagas diseases, using, for example, the skin test described above. In other embodiments polypeptides encoded by, expressed from, or originating from nucleotides of SEQ ID NOS: 1-96 may be employed. For example, individual polypeptides are chosen in such a way as to be complementary to each other. Examples of known T. Cruzi antigens which may be usefully employed in conjunction with the inventive polypeptides include TcF, TcSMT and/or CRA

For example, TcF is an antigen that is commercially available and may be used for detecting antibodies in Chagas disease patients. TcF comprises four tandem repeat proteins, namely TcD, TcE, B13/PEP-2, and TcLo1.2. (Burns, J. M., 1992. Proc. Natl. Acad. Sci. U.S.A. 89:1239-1243) (Houghton, R. L., 2000. J. Infect. Dis. 181:325-330). The serological reactivity of TcF, however, may vary from region to region.

The following examples are offered by way of illustration, and not by way of limitation:

### EXAMPLES

### Example 1

### Screening for Tandem Repeat Genes

DNA sequence data of Plasmodium falciparum 3D7 CDS version 2.1.4. (without pseudogenes); Leishmania major CDS version 5.2; L. infantum CDS version 3.0; and T. brucei Tb927_CDSs_v4_nopseudo, were obtained from GeneDB (www.genedb.org).

Also obtained were T. cruzi Annotated CDS Release 5.1 from TcruziDB (www.tcruzidb.org/tcruzidb); Toxoplasma gondii Annotated CDS Release 4.2 from ToxoDB (www.toxodb.org/toxo); Paramecium tetraurelia CDS v1.17 from ParameciumDB (paramecium.cgm.cnrs-gif.fr); Candida albicans orf coding assembly 21 from The Candida Genome Database (www.candidagenome.org); Mycobacterium tuberculosis Release R7 (34) from TubercuList (genolist.pasteur.fr/TubercuList/); and Salmonella enterica serovar Typhi CT18 (35) and Homo sapiens Hs36.2 CCDS nucleotide 20070227 from the NCBI database (www.ncbi.nlm.nih.gov/projects/CCDS/).

Tandem Repeats Finder, a program to locate and display tandem repeats in DNA sequences, was used to screen and analyze DNA sequences to find tandem repeats (tandem.bu.edu/trf/trf.html). The program calculates a score according to selected characteristics of the tandem repeat genes such as the period size of the repeat (i.e., the length of the repeat unit), the number of copies aligned with the consensus pattern, and the overall percentage of matches between adjacent tandem repeats.

A high score indicates that a gene possesses a large tandem repeat domain. The genes were regarded as tandem repeat genes if the scores from the Tandem Repeats Finder analysis were 150 or higher. The cutoff value of 150 may be used because such a value, among other things, is likely to eliminate genes with repeat domains shorter than 75 bp. When more than one tandem repeat domain was found within a gene, only the domain with the highest score was listed or used for further analyses and protein production.

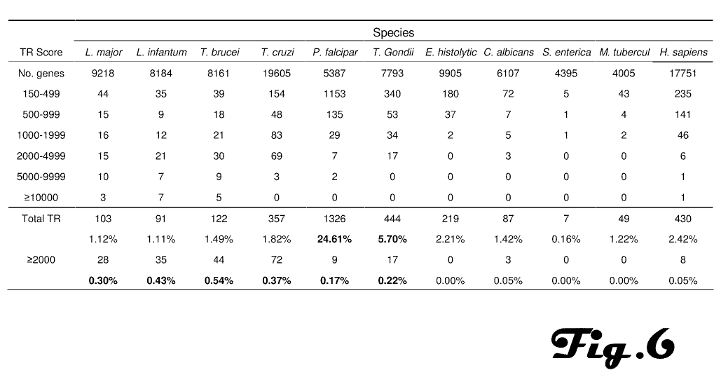

FIG. 6 is a table depicting the results of screening the above mentioned organisms for tandem repeat sequences. Results are broken down based on Tandem Repeat Finder score.

### Example 2

### Screening for T. Cruzi Tandem Repeat Genes

DNA sequence data of T. cruzi was obtained from GeneDB (www.genedb.org). Tandem Repeats Finder was used to screen, analyze, and score DNA sequences to find tandem repeats. A high Tandem Repeats Finder score suggests that the gene in question possesses a large tandem repeat sequence and that the repeat is highly conserved among the copies. Genes were regarded as tandem repeat candidates if the score from the Tandem Repeats Finder analysis was 500 or greater. The cutoff value of 500 was used because such a threshold score is likely to eliminate genes with a 250 bp-long or smaller tandem repeat domain.

By this computational analysis, 357 of 19,605 T. cruzi genes (i.e. 1.82%) were identified as being genes containing tandem repeat regions by the analysis using Tandem Repeats Finder based on a threshold score of 150 (See FIG. 4). Sequence analysis revealed that all 357 identified genes encode tandem repeats in amino acid sequence. The prevalence of tandem repeat genes in T. cruzi was found to be similar to those of other trypanosomatids and was not remarkably higher than other species.

However, when considering tandem repeat genes with a Tandem Repeat Finder score of 2000 or greater, whose tandem repeat domain is likely to be 1000 bp-long or larger, the prevalence of such genes was higher in L. major, L. infantum, T. brucei, T. cruzi, Plasmodium falciparum and Toxoplasma gondii than the protozoan parasite Entoamoeba histolytica, fungus Candida albicans, bacteria Salmonella enterica and Mycobacterium tuberculosis or mammal Homo sapiens. In particular, the trypanosomatid parasites were rich in large tandem repeat genes, with higher mean and median tandem repeat scores, and with higher prevalence of tandem repeat genes with a Tandem Repeat Finder score of 2000 or greater.

### Example 3

### Analysis of T. cruzi Tandem Repeat Genes

The biochemical properties of the selected T. cruzi tandem repeat proteins were analyzed for (1) a protein's molecular mass, isoelectric point, hydorophobicity, presence of a signal sequence and a trans-membrane domain; (2) the protein's known antigenicity and/or functions by Blast searches using both DNA and deduced amino acid sequences against the NCBI database, and (3) a mass-spectrometric evidenced protein expression profile, available through the database TcruziDB.

Biochemical characteristics such as average hydrophobicity, isoelectric point, and molecular mass were calculated using the ProteinMachine™ software package from Protein Advances (Protein Advances, Inc., Seattle, Wash.). To analyze the entire database, a software interface programmed in C# created protein data files as comma separated values for export to Excel. Average hydrophobicity/hydrophilicity plots of each sequence were determined using a modified Kyte/Doolittle algorithm with scores ranging from 0.6 (most hydrophilic score possible) to 9.0 (most hydrophobic score possible). Selected T. cruzi tandem repeat genes were analyzed for their specificity for T. cruzi, i.e., whether a homologous gene or protein is found in Leishmania or other organisms, by Blasting the DNA and deduced amino acid sequences against the NCBI database and GeneDB.

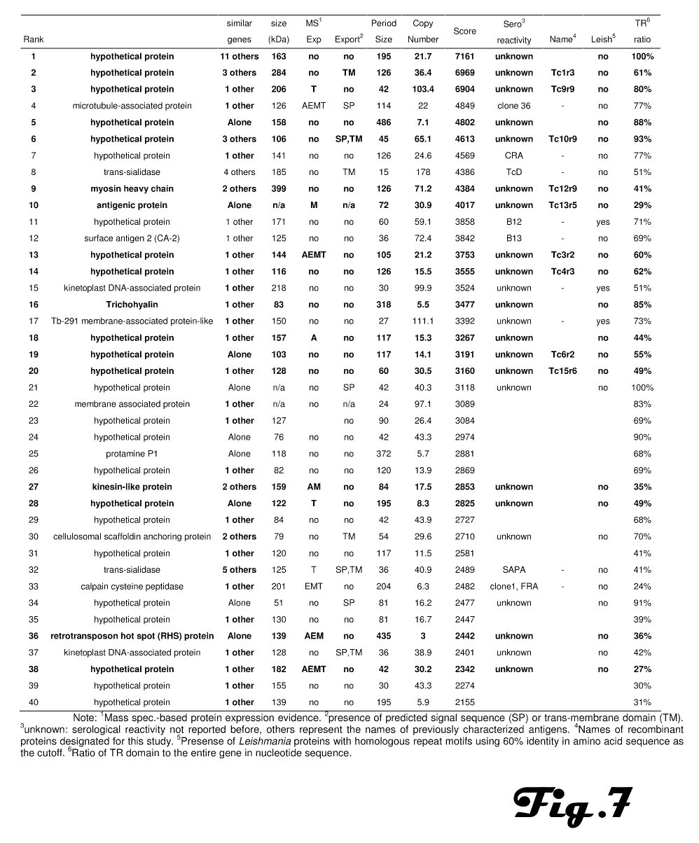

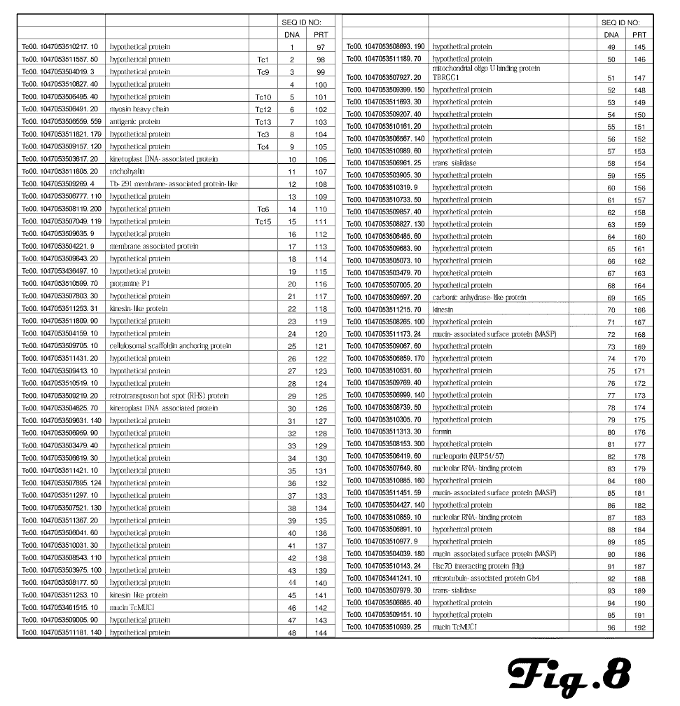

Additionally, an analysis was made as to the number of these T. cruzi tandem repeat proteins that have been previously characterized as antigens. There were sometimes multiple genes encoding tandem repeats with high similarity. For example, five individual genes were found as encoding the TcD repeat sequence. After consolidating 203 genes containing tandem repeats with a score of 500 or higher, based on 70% or greater identity in amino acid sequences, 106 genes with different tandem repeat sequences were identified. For example, the top 40 genes are depicted in FIG. 7. Among these selected 106 genes, 10 were previously characterized genes encoding antigenic repeat motifs including clone 36, CRA, TcD, B12, B13, SAPA, FRA, TcLo1.2, TcE and antigen 38, with the remaining 96 genes being previously uncharacterized as encoding antigens (see FIG. 8).

(Burns, J. M., 1992. Proc. Natl. Acad. Sci. USA 89:1239-1243) (Gruber, A., 1993 Exp. Parasite 76:1-12) (Ibanez, C. F 1988 Mol Biochem Parasitol 30:27-33) (Lafaille, J. J., 1989 Mol Biochem Parasitol 35:127-136) (DaRocha, W. D., 2002. Parasitol Res 88:292-300) (Affranchino, J. L., 1989. Mol Biochem Parasitol 34:221-228) (Houghton, R. L., 1999. J Infect Dis 179:1226-1234).

### Example 4

### Expression of T. cruzi TR Proteins

Partial tandem repeat domains containing multiple repeat units were either PCR-amplified or synthesized. Partial TR domains of Tc00.1047053510827.40 (Tc2) (SEQ ID NO: 4), Tc00.1047053511821.179 (Tc3) (SEQ ID NO: 8), Tc00.1047053509157.120 (Tc4) (SEQ ID NO: 9), and Tc00.1047053508119.200 (Tc6) (SEQ ID NO: 14) were amplified by PCR with T. cruzi total DNA using primer sets as following, Tc2: 5′ SEQ ID NO: 193, 3′ SEQ ID NO: 194; Tc3: 5′ SEQ ID NO: 195, 3′ SEQ ID NO: 196; Tc4: 5′ SEQ ID NO: 197, 3′ SEQ ID NO: 198 Tc6: 5′ SEQ ID NO: 199, 3′ SEQ ID NO: 200.

Partial TR domains of Tc00.1047053511557.50 (Tc) (SEQ ID NO: 2), Tc00.1047053510217.10 (Tc8) (SEQ ID NO: 1), Tc00.1047053504019.3 (Tc9) (SEQ ID NO: 3), Tc00.1047053506495.40 (Tc10) (SEQ ID NO: 5), Tc00.1047053506491.20 (Tc12) (SEQ ID NO: 6), Tc00.1047053506559.559 (Tc13) (SEQ ID NO: 7), and Tc00.1047053507049.119 (Tc 5) (SEQ ID NO: 15), were synthesized by Blue Heron Biotechnology, Inc. (Bothell, Wash.).

The amplified PCR products or synthesized oligonucleotides were inserted in-frame with the 6× His tag of vector pET-28a. The vectors were then transformed into the E. coli Rosetta strain. The transformed E. coli were grown in 2× YT medium, and expression of the recombinant proteins was induced by cultivation with 1 mM isopropyl-β-D-thiogalactoside for three hours. After lysing cells by sonication and centrifuging at 10,000×g, the supernatants were used for purifying the proteins as 6× His-tagged proteins using Ni-NTA agarose (Qiagen Inc., Valencia, Calif.). Proteins were bound to the resin, washed with sodium deoxycolate-containing buffer and eluted with buffer containing 250 μM imidazole. The eluted protein was dialyzed against PBS (pH 7.4), and the concentration of the purified protein measured by BCA protein assay (Pierce Biotechnology Inc., Rockford, Ill.). Purity of the proteins was assessed by Coomassie blue-staining following SDS-PAGE.

### Example 5

### Sero-Reactivity Analysis of Expressed T. cruzi Proteins

The expressed T. cruzi TR proteins were analyzed for sero-reactivity using sera from Brazilian or Ecuadorian Chagas disease patients (n=24). Sera from Brazilian visceral leishmaniasis (VL) patients (n=16) and healthy Brazilian people were used as controls. Proteins were diluted in ELISA coating buffer, and 96-well plates were coated with 200 ng of individual recombinant antigens followed by blocking with phosphate-buffered saline containing 0.05% Tween-20 and 1% bovine serum albumin. Plates were incubated sequentially with human serum samples (1:200 dilution) and with horseradish peroxidase-conjugated anti-human IgG (Rockland Immunochemicals, Inc., Gilbertsville, Pa.). The plates were developed with tetramethylbenzidine peroxidase substrate (Kirkegaard & Perry Laboratories, Gaithersburg, Md.) and scanned by a microplate reader at 450 nm (570 nm reference). Three additional recombinant proteins were tested as controls: T cruzi sterol 24-c methyltransferase (TcSMT) as a conserved antigen between Trypanosoma and Leishmania species; rK39 as a Leishmania-specific TR antigen; and CRA as a T. cruzi-specific TR antigen.

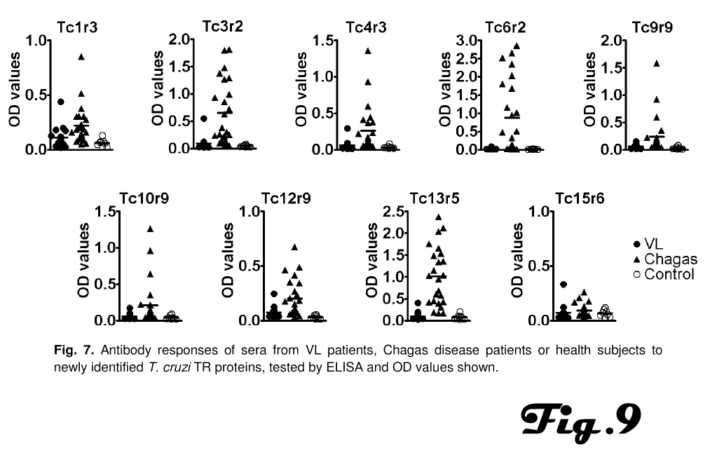

FIG. 9 depicts antibody responses of sera from visceral leishmaniasis patients, Chagas disease patients and healthy subjects to selected T. Cruzi tandem repeat proteins, which have been tested by ELISA.

### Example 6

### Use of Complementary Tandem Repeat Antigens

As described herein, TcF is an antigen that is used for detecting antibodies in Chagas disease patients, which comprises four tandem repeat proteins. The serological reactivity of TcF, however, varies from region to region.

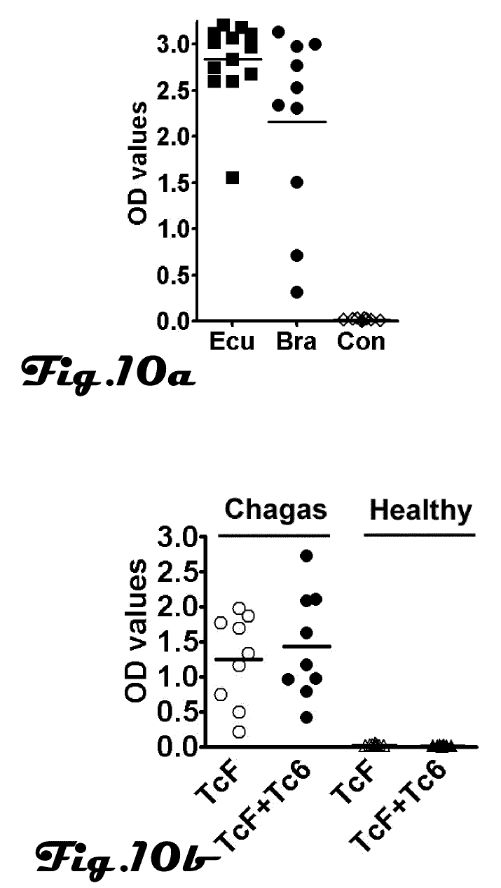

FIG. 10a depicts the reactivity of TcF in Ecuador and Brazil. Sera from Ecuadorian (Ecu) or Brazilian (Bra) Chagas patients and Brazilian healthy endemic controls (Con) were examined compared to the reactivity to a diagnostic fusion antigen TcF by ELISA.

FIG. 10b depicts the reactivity of the combination of Tc6 and TcD compared to the reactivity of TcF alone. As depicted in FIG. 10b, the combination of Tc6 and TcD improves the performance in an ELISA test, compared to TcF alone. Sera from Brazilian Chagas disease patients having relatively low response to TcF were examined compared to the reactivity to TcF alone or a mixture of TcF and Tc6 by ELISA. Sera from healthy subjects from the endemic areas are used as controls.

### CONCLUSION

Although specific embodiments have been illustrated and described herein, it will be appreciated by those of ordinary skill in the art and others, that a wide variety of alternate and/or equivalent implementations may be substituted for the specific embodiment without departing from the scope of the embodiments described herein. This application is intended to cover any adaptations or variations of the embodiment discussed herein. While various embodiments have been illustrated and described, as noted above, many changes may be made without departing from the spirit and scope of the embodiments described herein.

1. A fusion protein comprising at least a first and second tandem repeat unit,
wherein the first tandem repeat unit comprises an amino acid sequence having at least 8 consecutive amino acids of, and at least 70% homology to, an amino acid sequence selected from the group consisting of SEQ ID NO: 97-192; and
wherein the second tandem repeat unit comprises an amino acid sequence having at least 8 consecutive amino acids of, and at least 70% homology to, an amino acid sequence selected from the group consisting of SEQ ID NO: 97-192.

2. The fusion protein of claim 1,
wherein the first tandem repeat unit comprises an amino acid sequence having at least 8 consecutive amino acids of, and at least 70% homology to, an amino acid sequence selected from the group consisting of SEQ ID NO: 98-99, 101-105 and 110-111; and
wherein the second tandem repeat unit comprises an amino acid sequence having at least 8 consecutive amino acids of, and at least 70% homology to, an amino acid sequence selected from the group consisting of SEQ ID NO: 98-99,101-105 and 110-111.

3. The fusion protein of claim 1, comprising at least a first and second tandem repeat unit plurality,
wherein the first tandem repeat unit plurality comprises an amino acid sequence having at least 8 consecutive amino acids of, and at least 70% homology to, an amino acid sequence selected from the group consisting of SEQ ID NO: 97-192; and
wherein the second tandem repeat unit plurality comprises an amino acid sequence having at least 8 consecutive amino acids of, and at least 70% homology to, an amino acid sequence selected from the group consisting of SEQ ID NO: 97-192; and
wherein said first and second tandem repeat plurality are adjacent.

4. The method of claim 1 wherein the first tandem repeat unit and second tandem repeat unit are identical.

5. The method of claim 1 wherein the first tandem repeat unit has at least 8 consecutive amino acids of, and at least 70% homology to, the second tandem repeat unit.

6. The method of claim 1 wherein the first tandem repeat unit and second tandem repeat unit are adjacent and separated by a linker sequence.

7. An isolated polynucleotide that encodes a polypeptide which is selected from the group consisting of:
a polypeptide that comprises at least one tandem repeat unit, wherein the tandem repeat unit comprises an amino acid sequence having at least 8 consecutive amino acids of, and at least 70% homology to, an amino acid sequence selected from the group consisting of: SEQ ID NO: 97-192;
a polypeptide that comprises at least two tandem repeat units, wherein each tandem repeat unit comprises an amino acid sequence having at least 8 consecutive amino acids of, and at least 70% homology to, an amino acid sequence selected from the group consisting of: SEQ ID NO: 97-192, and
a polypeptide that comprises a fusion protein comprising at least a first and second tandem repeat unit, wherein the first tandem repeat unit comprises an amino acid sequence having at least 8 consecutive amino acids of, and at least 70% homology to, an amino acid sequence selected from the group consisting of SEQ ID NO: 97-192, and wherein the second tandem repeat unit comprises an amino acid sequence having at least 8 consecutive amino acids of, and at least 70% homology to, an amino acid sequence selected from the group consisting of SEQ ID NO: 97-192.

8. The isolated polynucleotide of claim 7 that encodes a polypeptide which is selected from the group consisting of:
a polypeptide that comprises at least one tandem repeat unit, wherein the tandem repeat unit comprises an amino acid sequence having at least 8 consecutive amino acids of, and at least 70% homology to, an amino acid sequence selected from the group consisting of: SEQ ID NO: 98-99,101-105 and 110-111;
a polypeptide that comprises at least two tandem repeat units, wherein each tandem repeat unit comprises an amino acid sequence having at least 8 consecutive amino acids of, and at least 70% homology to, an amino acid sequence selected from the group consisting of: SEQ ID NO: 98-99,101-105 and 110-111, and
a polypeptide that comprises a fusion protein comprising at least a first and second tandem repeat unit, wherein the first tandem repeat unit comprises an amino acid sequence having at least 8 consecutive amino acids of, and at least 70% homology to, an amino acid sequence selected from the group consisting of SEQ ID NO: 97-192, and wherein the second tandem repeat unit comprises an amino acid sequence having at least 8 consecutive amino acids of, and at least 70% homology to, an amino acid sequence selected from the group consisting of SEQ ID NO: 98-99, 101-105 and 110-111.

9. The polynucleotide of claim 7, comprising a nucleotide sequence having at least 24 consecutive nucleotides of, and at least 70% homology to, a sequence selected from the group consisting of: SEQ ID NO: 1-96.

10. A recombinant expression vector comprising a polynucleotide according to claim 7.

11. A host cell transformed with an expression vector according to claim 7.

12. A diagnostic kit for detecting T. cruzi infection in a biological sample, comprising:
a plurality of polypeptides, wherein each of the plurality of polypeptides comprises at least one tandem repeat unit, wherein the tandem repeat unit comprises an amino acid sequence having at least 8 consecutive amino acids of, and at least 70% homology to, an amino acid sequence selected from the group consisting of: SEQ ID NO: 97-192; and
a detection reagent.

13. The diagnostic kit of claim 12, further comprising a polypeptide comprising at least 8 consecutive amino acids of, and at least 70% homology to, an amino acid sequence selected from the group consisting of: SEQ ID NO: 98-99, 101-105 and 110-111.

14. The diagnostic kit of claim 12, further comprising a solid support, wherein said plurality of polypeptides is bound to a solid support.

15. The diagnostic kit of claim 14 wherein said plurality of polypeptides is non-covalently bound to a solid support.

16. The diagnostic kit of claim 15 wherein the solid support comprises one of nitrocellulose, latex and a plastic material.
